(19) United States
(12) Patent Application Publication
(10) Pub. No.: US 2024/0180150 A1
(43) Pub. Date: Jun. 6, 2024

(54) Green Closed Loop Bio-waste Refining Process For Producing Smart Active Extracts and Delivery Systems for Their Application

(71) Applicant: CELLACURE LLC, Atlanta, GA (US)

(72) Inventor: Anamaria ORZA, Atlanta, GA (US)

(73) Assignee: CELLACURE LLC, Atlanta, GA (US)

(21) Appl. No.: 18/041,298

(22) PCT Filed: Aug. 11, 2021
(86) PCT No.: PCT/US2021/045616

§ 371 Date: Feb. 10, 2023

Related U.S. Application Data
(60) Provisional application No. 63/064,135, filed on Aug. 11, 2020.

(51) Int. Cl. A01N 25/24; A01N 25/10; A01N 25/28; A01N 37/46; A01P 13/00; A01P 21/00; B82Y 5/00

(52) U.S. Cl. CPC A01N 25/24; A01N 25/10; A01N 25/28; A01N 37/46; A01P 13/00; A01P 21/00; B82Y 5/00

(57) ABSTRACT

The present invention relates to custom tailored bio-polymer compositions, nanoparticle bio-polymer compositions, green methods for making the bio-polymers and nanoparticle bio-polymers from bio-waste using DES/NADES, and agricultural formulations containing the bio-polymers and nanoparticle bio-polymers. The bio-polymers and nanoparticle bio-polymers can be used as biostimulants, used as targeted delivery systems or both. Bio-polymers and nanoparticle bio-polymers can be used to stimulate plant growth and development, stimulate root growth, increase adsorption of nutrients, increase crop yield, improve the quality and taste of crops, increase specific amino acid or other nutrient content, and for protection against various pathogens. Functional groups can be attached to the bio-polymers and nanoparticle bio-polymers to produce an engineered molecule with additional characteristics such as pH and/or temperature responsiveness, targeting or anchoring properties and improved stability. The method for synthesizing the bio-polymers and nanoparticle bio-polymers from bio-waste is simple, cost-effective and highly scalable for commercial availability. The bio-polymers and nanoparticle bio-polymers can be used in agricultural formulations such as fertilizers, pesticides, and herbicides.

### FIELD OF THE INVENTION

The invention pertains to a green and economically viable method for preparing agricultural formulations including but not limited to herbicides, pesticides, biostimulants and fertilizers, using natural deep eutectic solvents (NADES) to extract organic rich-waste biomass. The agricultural formulations utilize recovered and/or engineered, natural agricultural nutrients (hydrolyzed peptides, inorganic compounds, carbohydrates, lipids, micro/macro elements or a combination thereto) that are recycled from organic rich-waste resulting in less environmental contamination, fewer negative human health implications, more efficient utilization of energy and water, and economic prosperity.

### BACKGROUND OF THE INVENTION

In order to sustain the current food demand the agricultural sector (1) occupies approximately 40% of the total earth surface, (2) contributes to around 11% of the total global green gas emissions, and (3) comprises 70% of the global water usage for the irrigation of agricultural crops. Modern agricultural practices employ continuous and extensive management options to prevent disease and damage from pests and to manage plant growth and crop productivity. A necessary evil for industrial agriculture is the application of conventional chemical agricultural formulations protecting plants against thousands of species of plant pathogens, weeds, and insects that attack farm produce globally, as well as the application of growth promoting agents, taste improving agents, and physiological activity stimulating agents, to increase the yield and improve the quality of crops. The current agricultural strategies are costly and not sustainable as they result in environmental damage. In the United State alone, the economic loss from crop damage due to diseases and pests are estimated at several billion dollars annually. Efforts to combat pests and disease and improve plant productivity is estimated to cost additional billions annually. In addition, the excessive chemicals used to promote plant growth, manage diseases and pests and increase productivity lead to unintended environmental contamination with negative human health implications, as well as utilization of significant inputs of energy and water.

In the coming years, the rise in global population will drive an ever-increasing food demand that is expected to rise 70% by 2050; our agricultural practices must meet the challenges of feeding the global population, while simultaneously minimizing the consequences on human health and the environment and maintaining the ecological balance and conservation of soil biodiversity.

The existing state-of-the-art agricultural formulations result in highly inefficient delivery to the plants, thus requiring multiple applications and excessive quantities of chemicals. This excessive utilization of chemicals contaminates the environment and has negative implications for human and nonhuman biota. The chemical residues destroy the terrestrial ecosystem, the soil's microbial flora and natural humus, resulting in decreased soil porosity structure, nutrient availability, and an overall decrease in soil health and fertility. Additionally, unabsorbed chemicals are volatilized into the air causing air pollution and dissolved into water threatening the aquatic and costal marine ecosystem. In addition, the excessive application of chemicals increases their levels in plants possibly threatening the health of people and animals consuming the plants. Diseases such as cancer, obesity and endocrine disruption can be related to the consumption of foods that were produced using high concentrations of chemicals. In addition, the excessive exposure of farmers to toxic agrochemicals can cause long-term illnesses.

Unfortunately, in the last 40 years the quantities of agricultural chemicals applied per one hectare has increased dramatically. Pests and diseases have become resistant to the current chemical pesticide and fertilizer formulations; and a constant increase in concentration and in the number of applications is required to obtain the desired results.

It is necessary to develop green and effective methods of synthesis, innovative delivery technologies and sustainable, innovative agricultural agents. As potential raw materials, natural extracts, including but not limited to peptides, polysaccharides, lipids, and active inorganic compounds, can solve the above challenges and support sustainable agriculture. The natural extracts can be recovered from many municipal and industrial waste and by-product streams, particularly from food-related industries and processing plants and converted into valuable, innovative agricultural agents. One potential source is fish waste and by-products from the fish processing industry About 154 million tons of fish are caught or produced each year with a value of approximately $217.5 billion. About 70% of fish is processed before the final sale to a consumer, resulting in 20-80% of fish waste, depending on the level of processing and type of fish. Currently, due to their high protein content, a small part of the fish waste is used to produce fishmeal, fish oil and limited amounts of agricultural biostimulants. Unfortunately, the majority of the fish wastes are disposed of in the ocean causing environmental problems. Moreover, the organic components of the fish waste have a high biological oxygen demand and, if not managed properly, high oxygen demand poses environmental and human health problems. Similar problems are encountered for the disposal of plant by-products, and other animal or yeast organic-rich waste materials. The novel corona virus which causes COVID-19 has been linked epidemiologically to the Hua Nan seafood and wet animal wholesale market in Wuhan. John S Mackenzie, Microbiol Aust. 2020 Mar. 17: MA20013. Although the source of the pandemic is yet unknown, animal waste could be a potential source of zoonotic diseases.

Thus, it is essential to develop efficient, green, cost-effective methods for recovering and/or engineering active agricultural nutrients originating from organic-rich waste streams. Such methods will allow (1) the development of innovative agricultural formulations which will support a successful and sustainable agriculture, and (2) will help solve the global problem of waste disposal and by-product management, by mitigating their negative impact on the environment thereby providing the most sought after environmental protection and sustainability (Russ and Pittroff 2004). Recently, peptide hydrolysates derived from different waste sources were reported to be an important class of biostimulants able to increase plant growth and crop yield especially under environmental stress conditions. Giuseppe Colla Front Plant Sci. 2017; 8: 2202. Kenny Paul, nt Sci 2019 May 3; 10:493. Furthermore, various inorganic compounds recycled from plant biomass were showed to be effective natural defenses against insects and pest, with a safer and environmentally friendly profile. Yolice Tembo Front. Plant Sci., 28 Sep. 2018.

Peptide extracts (in their bulk form) from various organic rich-waste sources have been successfully embraced by the market and have received significant attention for their use as a biostimulant on a wide range of horticultural and agronomic crops, in both organic and conventional agricultural practices

Currently produced peptide extracts comprise free amino acids, soluble peptides, carbohydrates, phenols and limited amounts of plant nutrients and are generally applied by foliar application, however, root application and a combination of foliar and root application are also used. Peptide extracts have gained prominence as biostimulants because of their potential to modulate plant molecular and physiological processes. Peptide extracts enhance germination, plant growth, and alleviate the impact of abiotic stress on crops, resulting in increased crop yields and quality. Although, the mechanisms behind the beneficial effect of peptide extracts on plants is not fully known, it was recently attributed to their carbon and nitrogen components, which regulates carbon and nitrogen metabolism in plants, as well as nitrogen uptake and assimilation of key enzymes involved in the tricarboxylic acid cycle (citrate synthase, isocitrate dehydrogenase and malate dehydrogenase). Additionally, the application of peptides as biostimulants has been observed to interfere with plant hormonal mechanism and to promote auxin and gibberellin activity which enhances root and shoot growth, and thus improves crop productivity. Peptide extracts may also improve the availability of macronutrients and micronutrients in plants and enhance microbial activity in soil. Giuseppe Colla Front Plant Sci. 2017; 8: 2202.

Agricultural peptide extract formulations have been described for example in U.S. Application 2008/0302151A1 which discloses a water-soluble protein peptide fertilizer hydrolyzed from soy beans using proteolytic enzyme (protease) hydrolysis. U.S. Application 2019/0023750A1 discloses bioactive polypeptides produced by chemical modification for use in agricultural formulations. U.S. Pat. No. 10,252,950 discloses a process for releasing nutritional elements from plant and animal material using enzymes to produce a liquid hydrolysate. U.S. 2008/0134737A1 discloses a fertilizer composition containing a hydrolysate obtained from a by-product of an industrial fermentation process where the cell mass can be hydrolyzed using an acid. U.S. 20120129695 discloses a method of producing a plant biostimulant by hydrolyzing bacterial cells using an enzyme or acid hydrolysis to obtain a hydrolysate. EP2752399A1 discloses a method for obtaining a biostimulant fertilizer extract from brewing waste, using enzymatic hydrolysis combined with alkaline treatments (28% NHs to pH 9.6) at high atmospheric pressures and high temperatures. Before the present invention was made, the peptide extracts used for agricultural formulations were recovered from different waste sources using chemical or enzymatic processes and were used as fertilizers to improve plant growth and development.

Currently used extraction methods include chemical hydrolysis (acid or alkaline media), thermal hydrolysis, enzymatic hydrolysis and combinations thereof. Animal peptide sources are mainly hydrolyzed using chemical and/or thermal methods, which require toxic chemicals, and/or high temperatures and pressure, in some cases temperatures greater than 121° C. and pressure greater than 220.6 kPa. Due to the harsh conditions, some amino acids like tryptophan, cysteine, serine and threonine are partially or totally destroyed and many other amino acids are converted from the L-form to D-form (racemization) thus losing their biological activity and having a toxic effect on plants. Enzyme hydrolysis is generally more costly than chemical hydrolysis though the resulting peptide:free amino acids ratio, and the proportion of L-amino acids to D-amino acids is improved as compared to peptide extracts obtained by chemical hydrolysis. However, the peptide extraction yield using enzyme hydrolysis is not as high, likely due to the many purification steps which are required. In addition, enzyme hydrolysis consumes a significant amount of water and energy.

Peptide extracts can have phytotoxicity effects as well as suppression of growth due to a phenomenon known as general amino acid inhibition. General amino acid inhibition is due to excessive leaf uptake of free amino acids, which causes intracellular amino acid imbalance, energy drain due to active transport of amino acids, inhibition of nitrate uptake, and an increase of cell susceptibility to apoptosis. These free amino acids, and the formation of toxic compounds are a result of the peptide extraction process.

In order to maximize the beneficial effects of peptide extracts as biostimulants, advanced extraction methods are needed to avoid degradation of the peptide structure and the formation of toxic compounds, and to obtain high yields of pure peptides in a cost-effective way. In addition, methods for engineering peptides are needed to allow the peptide extracts to be used for purposes other than as biostimulants, such uses include but are not limited to herbicides, insecticides, and pesticides.

Novel extraction methods are needed to create targeted formulations with specific structures, physical chemical properties, and specific compositions of individual peptides, lipids, sugars and inorganic substances. The extraction methods must be cost effective, and result in extracts of high yield and purity. Moreover, with the continued awareness of environmental problems and the environmental consequences created both by the waste by-products and the current agricultural management practices, the extraction methods should also be green and scalable. Furthermore, in order to develop advanced agricultural formulations, the method should allow the newly recycled, natural agricultural nutrients to be engineered to enhance or add new properties and to increase the efficiency of their final application.

Peptide extracts can be combined with nano-materials in agricultural formulations or the peptide extracts can be engineered to nanoscale, and the resulting nanomaterial can be combined with other active agricultural materials to produce the desired agricultural active ingredient. The beneficial impact of nano-materials such as metals, metal oxides (silver, cerium, copper, silica, magnesium titanium and zinc oxide), or non-metals (chitosan and glycan) in conventional agricultural formulations or as a sole active agent have been reported in the literature, but the field is still very new. Silver, silica, aluminum oxide, titanium and zinc oxide have been used for their increased insecticidal activity, and copper oxide, silver, iron oxide, magnesium have been reported to be promising as herbicides. Although, it is well known that some metal based nano-materials are phytotoxic at high concentrations (>500 mg/l), at low concentrations (<50 mg/l) they present no signs of toxicity. From the non-metal class, bio-polymer nanoparticle materials, such as chitosan and β-D-glycans have been reported as beneficial for plant growth and disease prevention. The treatment efficiency varies depending on the: (1) nano-material type (metal oxide, non-metals, etc.), (2) formulation design (e.g. micro or macro formulation, where selectively large or small amounts of required nutrients are included), (3) nano-material concentration, (4) the application mode (foliar or root application), (5) plant species, (6) environmental conditions, and (7) experimental/exposure design (Greenhouse base experiments and a small number were report on field trials). Concerns regarding human inhalation and the negative effect on the environment and biota must be considered for any materials that are on a nanoscale. Methods for manipulating the toxicology of nanomaterials by controlling their physicochemical properties (size, shape, surface chemistry, and coatings) are addressed in the Handbook of Nanotoxicology, Nanomedicine and Stem Cell Use, edited by Saura C Sahu and Daniel A Casciano. The chapter “Impact of Various Nanosystems on Stem Cell Physiology” (Pages: 309-336) by A. Orza provides a comprehensive overview of the toxicological effects of various nanomaterials on human exposure. Additionally, Mutation Research/Genetic Toxicology and Environmental Mutagenesis, 2020, A. Orza, et al., provides new insights into the cytotoxicity and genotoxicity of cadmium oxide nanoparticles, their risk assessment, and toxicity on human exposure and the environment. Further, in Studia Universitatis Babes-Bolyai, Chemia, 2019 A. Orza, et al., report several computational studies for predictive toxicity assessment of various non-metal nano materials. This model can be used to modulate the physicochemical properties of non-metal nanoparticles in order to minimize, and perhaps overcome their human and environmental toxicity.

### SUMMARY OF THE INVENTION

The present invention provides a novel, sustainable, economical and eco-friendly green extraction method for recycling organic-rich bio-waste into beneficial formulations which can be used in many fields including but not limited to agriculture, pharmaceuticals (human and veterinary), cosmetics and food. The present invention provides a novel method for the extraction of biomass from various sources of waste using Natural Deep Eutectic Solvents (NADES), Deep Eutectic Liquids, Ionic liquids or a combination of NADES, Deep Eutectic Liquids, and/or Ionic liquids with ball-milling or/and sonication. This extraction method can be used to recycle waste from many industries. Custom tailored natural extracts can be obtained by using specific organic-rich waste, bio-waste or plant or animal materials, using specific NADES, changing the ratio between NADES and the peptide source, controlling the reaction temperature, and the application of ball milling or sonication. The natural extracts can include but are not limited to peptides, carbohydrates, polysaccharides, lipids, inorganic compounds, microelements, microelements, or a combination thereof. The present invention can be used to extract natural inorganic components with pesticidal and insecticidal properties to adapt them for use as natural agricultural pest and weed control products. The present invention can be used to control the size, shape, and physicochemical properties of bio-polymers in the natural extract, for instance peptides, lipids or polysaccharides. The extracted bio-polymers can be engineered to add new properties and functionalities such as high mechanical tensile strength, novel pH responsive properties, thermo-responsive properties, enzymatic responses, sonochemical properties, magnetic properties, paramagnetic properties, novel adherence properties, and/or novel plant physiological properties. The extracted bio-polymers are highly stable in aqueous solutions and can be used without further processing, in a solid or liquid form, or can be further engineered as targeted/non-targeted delivery systems in various agricultural and non-agricultural applications. The bio-polymers and delivery systems can be used in many non-agricultural applications including but not limited to cosmetics and cosmeceuticals, perfumery, food industry, biofuels and in many other technological applications. They can be used as active components and/or delivery systems in commercial and consumer formulations and products.

### BRIEF DESCRIPTION OF THE DRAWINGS

FIG. 1A-FIG. 1L. Show that DES can be customized according to the final need and final reaction yield. Their properties can be tailored by changing the composition and ratio of the starting compounds. FIGS. 1A-C show that effective extraction of a keratin product from chicken feathers can be tailored using different compositions and ratios of DES, such as; Urea: K2S (FIG. 1A); Urea:NaSH, (FIG. 1 B); and Urea:Na2S (FIG. 1C). FIG. 1D shows the impact of the precipitation method and its use as a solvent on keratin purity and structure. In accordance with, FIGS. 1A-D, FIG. 1E, confirms that the extraction of various keratins of different molecular weights and purities can be tailored by DES composition, molar ratio, and precipitation solvent.

FIGS. 1G-I show that the choice of DES can affect the structure of the extracted keratin. FIG. 1F, shows the impact of NADES methods on the reaction yield.

FIG. 2A-FIG. 2K Show that the method according to the present invention can be applied to any type of organic-rich bio-waste to extract a desired biomass. Additionally, it shows that different types of DES can be customized and chosen to improve the extraction efficiency of the desired compound, from proteins to polysaccharides to flavonoids to colorants. FIGS. 2A-D show the extraction of chitin from shrimp shells, FIGS. 2F-I show the extraction of cellulose from wood. FIG. 2J shows the extraction of a flavonoid from the root of Virginia Creeper; and FIG. 2K shows the extraction of a colorant.

FIG. 3A-FIG. 3P Show that the current method can produce various delivery systems and incorporate active ingredients. It also shows that the delivery system size and shape can be tailored by choosing the appropriate functionality—oleyl amine, ca-stearate, PEI were selected, and the TEM images confirm the shape, encapsulation and size in FIGS. 3A-P and the precipitating solvents. A schematic representation of the reaction steps and procedures is also presented on the top Panel.

FIG. 4A-FIG. 4D Show that a eutectic occurs at a defined ratio of compounds. Urea:Na2S; Urea:NaSH; ChCl:EG, and ChCl: oxalic acid are shown. A deep eutectic is mainly caused by shielding the charge of the anion by complexing it with hydrogen bond donors.

FIG. 5A-FIG. 5P Show that different methods and functionalities produce different shapes and sizes of delivery systems. They show that the method of the invention can be tailored to produce targeted delivery systems in accordance with needs of the final application.

FIG. 6A-FIG. 6J Show that using the present invention, a vast diversity of products can be extracted. The processes are versatile and can be applied to manipulate the size and shape of the extract and the form of the delivery systems. FIG. 6A-FIG. 6J show that chitin nanoscale delivery systems of different sizes and shapes, can encapsulate active herbicide ingredients and be used as targeted delivery systems.

FIG. 7A-FIG. 7G Show that a highly effective delivery system can be produced with an increased drug loading capacity of 3× to 10× and even 16× more drug than the polymer, in a weight ratio (FIG. 7 Top Panel). The products of the invention and the delivery systems of different sizes and compositions possess control release properties such as pH responses, enzymatic responses and some chemical responses.

FIG. 8A-8D Show a particular example of an herbicide delivery system that is targeted and has pH and enzymatic control release profiles. The release profile was demonstrated using TEM, where the leaching of the drug and swelling of the responsive polymer is slow at pHs under 7.3.

FIG. 9A-FIG. 9R Show that the products of the invention can be used in agricultural applications, they penetrate the leaf, internalize into the plant after both foliar and root application, they translocate in a bidirectional manner, and distribute the active ingredient all over the plant. The methods of the invention and procedure and products can be used to form smart formulations for superior agriculture. The expanding implementation of nanotechnology for delivering fertilizer, herbicides, pesticides, DNA, RNA, a protein or a combination thereof, to treat, modify, protect, and improve the agricultural crops for increasing yield is demonstrated.

### DETAILED DESCRIPTION OF THE INVENTION

A eutectic system is a system is formed by combining at least two components that solidify at lower temperature than either one of them in a pure state. A deep eutectic solvent (DES) is a type of ionic solvent comprised of a mixture of at least two components that has melting point much lower than either of the individual components. Natural Deep Eutectic Solvents (NADES) are a class of DES that is composed of natural, non-toxic components. They are a relatively new class of green solvents that have recently attracted attention as solvents for the pre-treatment and extraction of biomass from waste material. NADES is used herein to include both the singular “solvent” and the plural “solvents”, and in some cases, will also refer to deep eutectic system(s). At other times, the term “deep eutectic solvent system” may be used, including “DES system”. Those of ordinary skill in the art will recognize the different uses in context.

The present invention combines the benefits of using DES/NADES, to obtain high purity and high yields of bio-polymers at low cost using environmentally friendly non-toxic chemicals, with the power of ball milling, and/or sonication in order to increase extraction yields, manipulate the size, shape, molecular mass, and composition of the extracted bio-polymers, reduce extraction time, and lower the quantity of NADES required to produce the extracted bio-polymers. Wet and/or dry chemical techniques can be applied to engineer the extracted product by adding at least one functionality, to manipulate size and/or shape, to change the composition of the bio-polymer and its composite, to add novel properties, or/and to create delivery systems of nano or micro scale. Delivery systems can be engineered to transport active ingredients or a combination of active ingredients of various sizes having various properties, and release the active ingredients to a specific site. The delivery system can contain molecules that respond to outside stimuli such as a change in pH, an enzymatic response, and/or a magnetic response. The delivery system can be a targeted and controlled release delivery system.

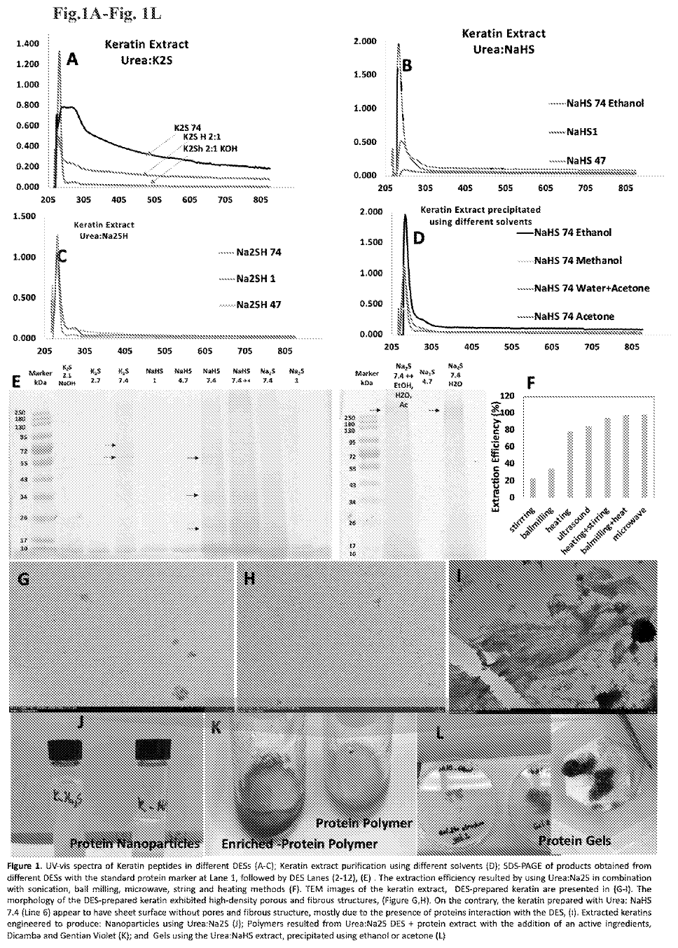

In one embodiment, Keratin protein can be extracted from chicken feather waste. The keratin extract size, shape, composition, and purity can be manipulated by choosing different DES components, molar ratios, and precipitation solvents (see FIGS. 1A-L). UV-VIS Spectra of the extracts shows the extraction of keratins with different properties (see FIG. 1A which shows the results of the following combinations Urea: K2SH molar ratio 2:1; Urea: K2SH, molar ratio 2:1 with the addition of KOH, and Urea: K2SH molar ratio 1.2 g+74 g). The peak shift demonstrates the extraction of keratins with different properties. Urea: K2SH molar 2:1 has an absorption peak at 300 nm compared with Urea: K2SH molar ratio (1:0.09) that absorbs at around 250 nm. Similarly, by manipulating the components of DES, various molecular weights, can be extracted. SDS-PAGE analysis can be used to confirm the extraction of different molecular weight keratins, such as around 10 KDa for both Urea: K2SH 2:1 (FIG. 1 E, Lane 2) and Urea: K2SH, molar ratio 2:1 with 0.5 M of KOH (FIG. 1E, Lane 1) and three different molecular weight keratins for Urea: K2SH (7.4 in the figures means 1:0.09) (FIG. 1 E, Lane 3). FIG. 1 E, Lane 12, shows a keratin extract of around 250 KDa extracted using Urea: Na2S while Lane 3, shows a 10 KDa extract of Urea: K2SH (1:0.09). FIG. 1F, shows the manipulation of reaction efficiency by choosing the appropriate extraction method.

The size, composition and shape of the product can also be manipulated by changing the DES and reaction conditions. The manipulation of the size and shape of one embodiment is shown in FIGS. 1 F-H, where FIGS. 1 F-G show the transmission electron microscopy images of extracts obtained using Urea: K2SH (2:1) and Urea: K2SH (2:1+KOH). FIG. 1H shows the product obtained from Urea: Na2S (1:0.09).

Nanoparticles, polymers, enriched polymers, and gels can be obtained by manipulating the DES and reaction conditions. FIG. 1J, shows nanoparticles produced by extraction using Urea: Na2S for 2 h, at 60° C., with the further addition of water, and without removing the DES. FIG. 1H, shows the polymer or enriched polymer of a keratin product with the addition of Dicamba herbicide and gentian violet. FIG. 1L shows the different protein gel products that can be obtained using Urea: NaHS DES and precipitation with ethanol or acetone.

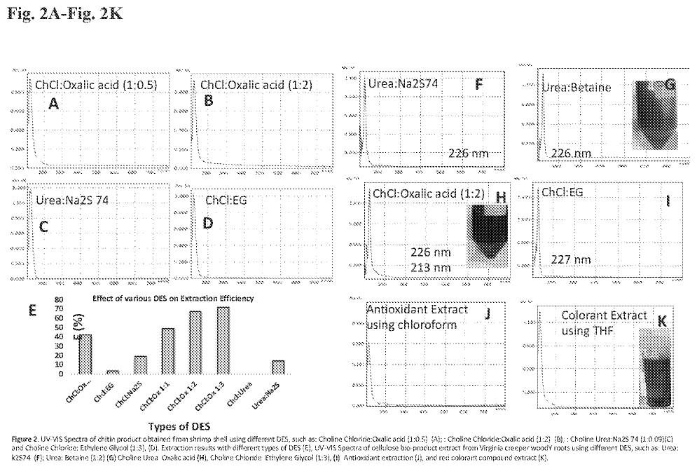

The method according to the present invention can be applied to any type of organic-rich bio-waste in order to extract a desired biomass. For example, chicken feathers (see FIG. 1 which shows the extraction and analysis of a keratin product from chicken feathers), crab shells (see FIG. 2A-D which shows the chitin product analysis obtained from crab shells), and wood roots (see FIG. 2F-I which shows the extraction of cellulose from woody roots of Virginia Creeper). FIGS. 2J-K show Virginia Creeper active compounds and FIG. 2J shows the extraction of flavonoids and FIG. 2K shows colorants). The method according to the present invention can be carried out as a one pot synthesis in which the NADES is made and the bio-polymers are extracted from bio-waste without isolating the NADES before the extraction, or removing the NADES after the extraction. The method can also be carried out as a multi-step process depending on the desired formulation composition. The present invention provides a method for synthesizing sustainable, non-toxic, targeted hydrolyzed bio-polymer compositions and/or nanoparticle compositions for use as bioactive components, as a delivery system or both. Currently peptide extracts from bio-waste are only used as agricultural fertilizers. Using smart nanodelivery systems, nano ingredients such as bioactive nutrients, pesticides, insecticides, herbicides, polysaccharides, lipids, inorganic substances or a combination thereof, can be used to produce formulations that not only feed and enhance plant growth, but also protect the plant against pathogens.

NADES are bio-based deep eutectic solvents (DES or Low Transition Melting Mixtures LTMM) which are sustainable and have little negative environmental impact. NADES are non-toxic for both human health and the environment. NADES were originally prepared using combinations of common metabolites which are abundant in plants (Young Hae Choi, et al., Are Natural Deep Eutectic Solvents the Missing Link in Understanding Cellular Metabolism and Physiology?, Plant Physiology, August 2011, 156 (4) 1701-1705; DOI: 10.1104/pp. 111.178426). NADES have been linked to natural phenomena in plants such as drought resistance, protection against high salinity, cryoprotection and germination. Thus, when NADES are used to extract bio-polymers and other natural components from bio-waste for agricultural formulations, the extracted components do not need to be purified to remove the solvents. The present invention recycles bio-waste to obtain a biomass of proteins, polysaccharides, lipids, inorganic materials such as polyphenols, pigments, dietary fibers, and other valuable raw materials for various applications.

All of the ingredients for making NADES can be found in cells and different NADES may be formed in different cellular compartments adapted to the different kinds of compounds and proteins to be conserved when dissolved in NADES. NADES can be obtained by mixing two or more natural occurring compounds (alcohols, sugars, amino acids, natural acids, etc.), wherein at least one of the NADES components acts as a hydrogen bond donor (HBD) and at least one NADES solvent component acts as a hydrogen bond acceptor (HBA). In a preferred embodiment, the HBA is choline chloride or an amino acid, and the HBD is oxalic acid or glucose. NADES are almost non-volatile and cannot be removed by evaporation, are chemically and thermally stable, non-flammable and are good solvents for many organic compounds. NADES can be made by combining sugar mixtures, sugars and acids, acids and amino acids, acids and bases, and bases and sugars. NADES can also be made by combining different bio-wastes containing organic-rich sugars, acids, amino acids, and bases. NADES can be customized for the extraction of specific compounds by combining different components. The components can be selected from but are not limited to choline chloride, choline bitartrate, betaine, lactic acid, DL-malic acid, citric acid, phytic acid sodium, D/L-proline, L-serine, L-glutamic salt, D-(+)-glucose, D-(−)-fructose, and β-alanine, malonic acid, maleic acid, aconitic acid, L-(+) tartaric acid, glycol, 1,2-propanediol, glycerol, meso-erythritol, xylitol, ribitol, adonitol, D-sorbitol, D-xylose, A-L-thamnose, sorbose, D-mannose, D-(+)-galactose, sucrose, D-(+)-trehalose, maltose, raffinose, proline, inositol, oxalic acid, β-alanine, L-proline, urea and choline chloride.

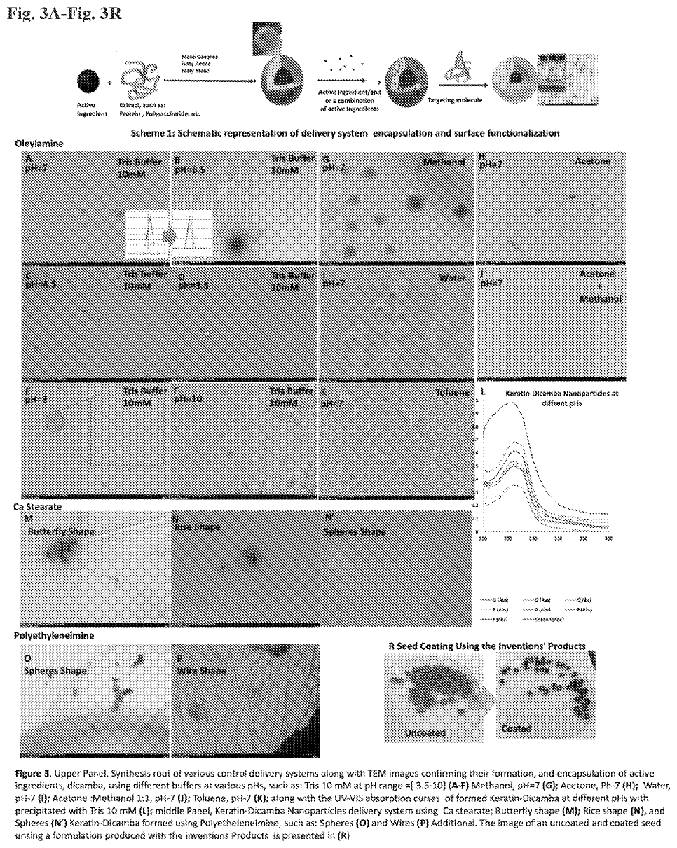

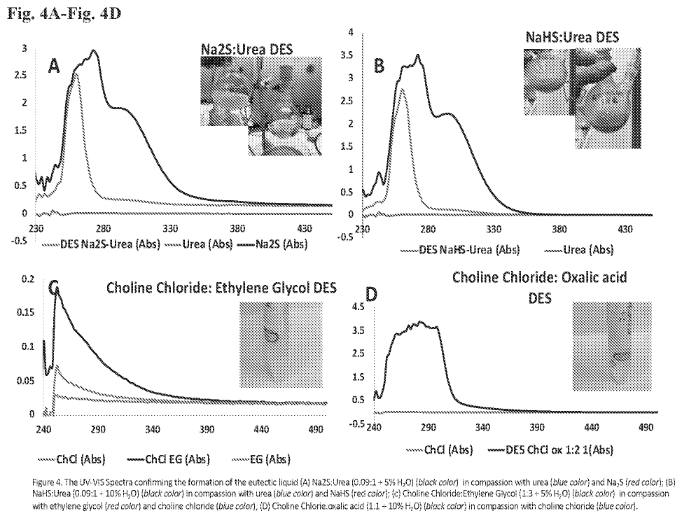

The DES and NADES are selected based on their basicity/acidity properties, composition and viscosity. The basicity/acidity of the HBD influence the size and shape of the final extract. Additionally, the temperature, reaction time, ball milling speed, sonication power, and microwave power can also be tailored to produce various classes of extracts. The extraction method can be used to control the extraction efficiency and also to manipulate the size, shape, encapsulation and functionalization of the extracted product. Several classes of DESs were established for their ability to directly extract products at nano and/or micro scale. Various sizes (1-1000 nm) and shapes (spherical, rods, wires, rhombic, cubes) can be obtained using DESs such as ChCl-Oxalic acid, ChCl-phosphotungstic acid (PTA), ChCl-acetic acid, ChCl-Lactic acid, ChCl-Ethylene glycol, ChCl-Glycerol, Urea:NaHS, Urea:Na2S and ChCl-Urea. See FIG. 3L which shows a keratin nanoproduct obtained using DES Urea:Na2S (1:0.09), ratio solid to DES 10:1 (wv/wv %), reaction time of 1.5 h, at 80° C., with the further addition of water and no DES removal. The extraction method can be used to obtain enriched polymers by adding an additional component into the reaction, where the additional ingredient interacts with the extract (purified or non-purified extract) and forms an enriched polymer. In one embodiment, keratin was extracted using ChCl: Na2S (1:0.09) DES for 1 h and precipitated using ethanol. A freeze dried extract was prepared and mixed in a weight ratio of keratin:Dicamba: GV of 1:3:0.5 (wv/wv %). The reaction was initiated by adding 5% H₂O to the dry mixed powder, followed by the addition of lipid to produce a keratin of 2-15% lipid modification, preferably 5-8% modification. The present invention can be used to produce nano/micro delivery systems of various sizes and shapes by adding the enriched polymer to different solvents and precipitating the particles (see FIGS. 3A-P). In one embodiment, a DES can be obtained by mixing solid urea with solid Na2S in a ratio (1:0.09) with 5-15% water, at 80° C. until liquid. FIG. 4 shows the formation of different DESs, such as Urea: Na2S, (A), Urea: NaHS (B), ChCl:EG 1:3 (C), and ChCl: oxalic acid 1:2 (D). In all cases the UV-VIS spectra show a new absorption peak that is shifted to the right at higher wavelengths as compared to the original component absorption peaks. In another embodiment, pure NADES can be obtained by mixing ChCl-Urea and ChCl-Thiourea DESs in a 1:2 or 1:1 molar ratio. The DESs are prepared by separately dissolving the urea (thiourea) and ChCl in water, then mixing and freeze drying to produce the DESs. In a further embodiment, the DESs are selected from a class of DES with high viscosity and high acidity, or basicity, and/or from a class of compounds that are active agricultural ingredients such as ChCl-succinic acid and ChCl-glucose; ChCl-citric acid, ChCl-malonic acid or oxalic acid, ChCl-glyoxylic acid}, ChCl-phosphonophormic acid, and ChCl-glucose-citric acid. NADES can be prepared from sugars and water, such as glucose, sucrose and/or fructose with 0%-20% water. Glucose, sucrose and/or fructose can be combined with a selected acid, such as citric acid or oxalic acid, or lactic acid, etc. to produce a NADES. The NADES can also be prepared from a sugar-rich-bio-waste and an acid-rich bio-waste (resulting from fermentation of the bio-waste).

The initial organic-rich bio-waste source is selected based on the desired agricultural application (e.g. plant growth, protection against pathogens, etc.). For example, the optimum bio-polymer to be used in connection with agricultural formulations can be determined based on the following criteria: 1) the intended targeting mechanism: a) direct plant internalization and stimulation, b) indirect stimulation through microbiota taxa, or c) a combination of direct and indirect stimulation; 2) the particular physiological mechanisms in the plants which will be stimulated (specific peptides and targeted amino acids are known to activate certain physiological mechanisms in plants); and 3) the role of the bio-polymerase bioactive agents, delivery systems or both.

The initial bio-polymer source can be bio-waste or other plant, yeast or animal derived materials. Plant sources useful for obtaining specific peptides include for example: Crambe (Crambe abyssinica), Canola, oilseed rape (Brassica napus and Brassica rapa) jojoba (Simmondsia chinensis), niger (Guizotia abyssinica), Sesame (Sesame indicum), Sunflower (Helianthus annuus), Groundnut (Arachis hypogaea), Soybean (Glycine max); Lupinus albus and Lupinus angustifolius, peas (Pisum sativum) and beans (Vicia faba), Forage peas, Lucerne (Medicago sativa), Bird's-foot trefoil (Lotus corniculatus), Red clover (Trifolium pratense), Sainfoin (Onobrychis viciifolia), White clover (Trifolium repens), Kudzu (Pueraria lobata), Sericea Lespedeza (Lespedeza cuneata); Aeschynomene, Arachis, Centrosema, C. macrocarpum, C. pubescens, Desmodium, Leucaena, Macroptilium and Stylosanthes. The initial bio-polymers can also be extracted from single cell protein sources such as: Active dry yeast, Brewers dried yeast Brewers liquid yeast, Grain distillers dried yeast, Primary dried yeast, Selenium yeast, Torula dried yeast or Candida dried yeast. Specific yeast sources include for example: Saccharomyces cerevisiae, Saccharomyces pastorianus, Saccharomyces eubayanus, Schizosaccharomyces pombe, Torulaspora delbrueckii, Lachancea thermotolerans, Saccharomycodes ludwigii, and Brettanomyces bruxellensis. Direct-fed microorganisms can also be used as a source for peptides, such as: Aspergillus figer, Aspergillus oiyzae, Bacillus coagulans, Bacillus lentus, Bacillus licheniformis, Bacillus pumilus, Bacillus subtilis, Bacteroides amylophilus, Bacteroides capillosus, Bacteroides ruminocola, Bacteroides suis, Bifidobacterium adolescentis Bifidobacterium animalis, Propionibacterium freudenreichii, Propionibacterium shermanii, Saccharomyces cerevisiae, Enterococcus cremoris, Enterococcus diacetylactis, Enterococcus faecium, Enterococcus intermedius Enterococcus lactis, Enterococcus thermophiles, Lactobacillus curvatus, Lactobacillus delbruekii, Lactobacillus fermentum, Lactobacillus helveticus, Lactobacillus lactis, Lactobacillus plantarum, Lactobacillus reuteri, Leuconostoc mesenteroides, Pediococcus acidilacticii, Pediococcus cerevisiae (damnosus), Pediococcus pentosaceus, Propionibacterium acidipropionici, Bifidobacterium bifidum, Bifidobacterium infantis, Bifidobacterium longum, Bifidobacterium thermophilum, Lactobacillus acidophilus, Lactobacillus brevis, Lactobacillus buchneri, Lactobacillus bulgaricus, Lactobacillus casei, Lactobacillus farciminis, and Lactobacillus cellobiuso. Animal sources useful for obtaining specific bio-polymers include for example: milk, cheese, and other dairy products, eggs, blood, gelatin, meat, various fish species, feathers, skin, and crustacean shells.

It is well known that individual peptides and amino acids specifically alter various plant physiological processes. In order to maximize the beneficial effect of the bio-polymer extracts, and to make the production more effective, it is necessary to select a target composition of bio-polymers that are responsible for a specific role in the plant (e.g. biostimulant or protection from biotic and abiotic stress). Therefore, the source of the bio-polymer and its compositions must be chosen to maximize their effectiveness in the plants.

The source of the bio-polymers is selected to produce natural extracts having targeted amino acids, which confer specific physico—chemical properties to the bio-polymers and the final bio-polymer can be tailored to activate a physiological mechanism. These natural extracts can be used in agricultural formulations for both plant growth and protection. When selecting the source for the bio-polymers and natural extracts, the following should be considered: 1) species and cultivar of the plant to be treated, 2) phenological stage of the plant, 3) growing conditions, and 4) application mode (delivery to leaves or roots). For example, specific classes of bio-polymers can be selected to be used in formulations that enhance and support seed germination. Peptides rich in γ-aminobutyrate (GABA), Arg, Glu and Met stimulate germination and early seedling growth. When seedlings are about 35 days old, arginine rich peptides are preferred. The bio-polymerscan be applied as a coating on seeds, a formulation applied with the seeds or applied after seeding, or a combination thereof. A process to select bio-polymers and specific compositions for agricultural applications is schematically presented in FIG. 1. The fundamental functions of various amino acids on plants are shown below in Table 2.

TABLE 2
Functions of various amino acids in plants

| Function | Amino acid type |
|---|---|
| Anti-stress agent | Hyp, Pro |
| Chelating agent | Cys, Glu, Gly, |
| | His, Lys |
| Cold weather resistance | Ala, Arg |
| Generative development of plants and improvement of | Hyp, Pro |
| the plant pollen fertility | |
| Growth stimulator | Glu |
| Precursor of auxin | Ser, Trp, Val |
| Precursor of chlorophyll | Gly |
| Precursor of polyamines: necessary to start the cell | Arg |
| division | |
| Precursor to the formation of lignin and woody tissues | Phe |
| Regulation of the water balance | Hyp, Pro, Ser |
| Reserve of organic nitrogen necessary for the synthesis | Glu |
| of other amino acids and proteins | |
| Stimulation of the chlorophyll synthesis | Ala, Lys, Ser |
| Stimulation of the ethylene synthesis | Met |
| Stimulation of the germination | Asp, Glu, Lys, |
| | Met, Phe, Thr |
| Stimulation of the hormone metabolism | Ala |
| Stimulation of the resistance mechanism to viruses | Ala |

The source for the bio-polymers and natural extracts is selected by considering the total amino acid concentration and the concentration of nitrogen N (%). A single source or a mixture of sources can be used to obtain the desired bio-polymers and natural extracts. The effect of a bio-polymer on a plant differs depending on the source of the bio-polymer. For example, collagen, keratin, and chitosan peptides could be extracted from fish and seafood waste, skin, hides and feathers. Ears, feet, lungs, and stomach tripe contain larger amounts of proline, hydroxy proline, and glycine, but lower levels of tryptophan and tyrosine. Chicken feathers are a good source of keratin peptides.

TABLE 4
Amino Acid (AA) Concentrations from Various Sources.

| | | | | | | N free |
|---|---|---|---|---|---|---|
| Item, % | Soybean | Blood | Feather | Poultry | Fish | AA % |
| Methionine | 1.29 | 0.6 | 0.6 | 1.0 | — | 9.4 |
| Cysteine | 1.25 | 0.5 | 4.3 | 1.0 | 3.00 | 13.6 |
| Lysine | 6.04 | 7.1 | 2.3 | 3.1 | 7.67 | 19.2 |
| Threonine | 3.85 | 3.2 | 3.8 | 2.2 | 3.63 | 11.8 |
| Isoleucine | 1.5 | 1.0 | 3.9 | 2.2 | 4. | 10.7 |
| Valine | 4.71 | 7.3 | 5.9 | 2.9 | 5 | 12 |
| Tryptophan | 7.51 | 1.3 | 0.6 | 0.4 | 1.33 | 13.7 |
| Arginine | 6.86 | 3.6 | 5.6 | 3.9 | 6.33 | 35.9 |
| Histidine | 3.06 | 3.5 | 0.9 | 1.1 | 1.67 | 27.1 |
| Leucine | 6.79 | 10.5 | 6.9 | 4.0 | 7.67. | 10.7 |
| Phenylalanine | 4.71 | 5.7 | 3.9 | 2.3 | 3.6. | 8.5 |
| Tyrosine | 3.66 | 2.1 | 2.5 | 1.7 | 3.5. | 7.7 |
| Glycine | 4.65 | 4.6 | 6.1 | 6.2 | | 18.7 |
| Serine | 4.91 | 4.3 | 8.5 | 2.7 | 6.00. | 13.3 |
| Aspartic acid | 10.46 | — | — | — | | |
| Glutamic acid | 16.25 | — | — | — | | |

The natural extract containing bio-polymers is prepared by 1) mixing the bio-waste solid by-product with NADES; 2) Increasing the temperature to about 40° C. to about 80° C. and mixing constantly at about 600 rpm/min to about 1200 rpm/min. Optionally, the resulting mixture can be filtered and precipitated with an organic solvent (preferably ethanol) then drying the precipitate by freeze drying or oven drying. The biomass prepared by the extraction process according to the present invention has an extraction yield between 45-98%, preferably between 92-98% and a purity level between 80-100%, preferably between 95-100%. The extraction efficiency, purity of the extracted biomass, and molecular weight of the extracted biomass can be modulated by changing parameters such as the DES or NADES components molar ratio, DES/NADES purity, the reaction temperature, and the solvent to solid ratio. The NADES of the present invention can be recycled and reused or can be included in the agricultural formulation without purification. The extracts obtained using this method are food grade and can be used in various applications without risk of toxicity.

The extraction process can be combined with other steps such as dry or wet chemical engineering by adding a functionality group, ball-milling, ultrasonication or microwaving to produce individual single-chain bio-polymer composites or chain folding nanoparticle composites. The process allows one to control the precursor chain (e.g. different bio-polymer molecular weights), add other molecules to the bio-polymers (e.g. bioactive agents), dictate the properties of the final nanoparticle (e.g. targeting, anchoring, etc.), and control the nanoparticle size and shape, thus, allowing for the production of distinct, custom molecules. Different sizes and shapes of nanoparticles encapsulated with dicamba are shown in FIG. 3, and atrazine is shown in FIG. 8.

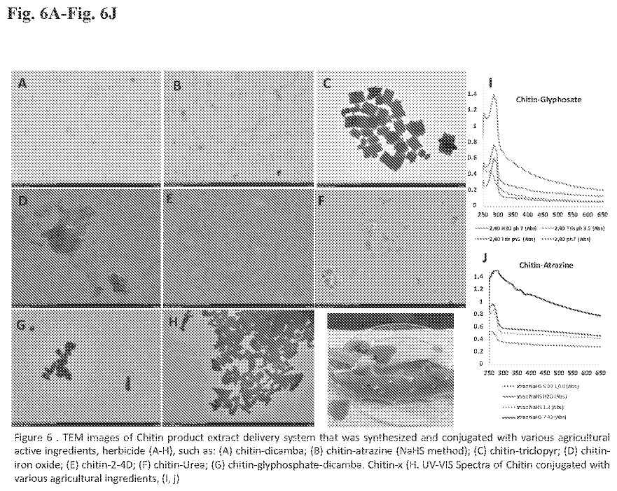

Extraction of the biomass with NADES combined with ball milling or ultrasonication produces small molecular weight peptides, microwires, nanowires and nanospheres. Ball milling is a mechanical process using a planetary ball milling at speeds between 50 to 800 rpm, used during the extraction with NADES. Ball milling combined with NADES extraction can result in higher yields, shorter extraction times and higher purity than a simple NADES extraction. Bio-polymer structural and physico-chemical properties can be modulated, for bio-polymers of various molecular weights, structures and compositions can be obtained. Using various rpm and different solvent with or without specific temperatures allows one to modulate the shape of the extracted bio-polymers, such as synthesis of nano and micro wires, and nano spheres, and possibly other shapes. Different shapes can be obtained by changing the surface energy of the molecule. Further, the addition of a secondary active ingredient, and its functionalization on the surface of the resulting nano bio-polymer or its encapsulation into the nanoparticles, or both can be achieved. When ultrasonication is used, the bio-polymers are hydrated in DES or NADES between 10 min and 3 hours, preferably for 30 minutes-1 hour, at temperatures between 40-120° C., followed by sonication between 150 W to 800 W for 3 minutes to about 45 minutes, but most preferably between 3 min to 20 min to produce the nano bio-polymers. When using DES or NADES for the extraction of the bio-polymers, sonication can be performed in the same liquid, during or after the bio-polymer extraction. Sonication allows the formation of nanoparticles and microparticles and it assists in both the particle surface functionalization and encapsulation, and reorientation of the nanoparticles. For instance, a bioactive compound can be encapsulated by adding the bioactive agent and sonicating again. The bioactive compound can also be released by exposing the encapsulated nanoparticles to sonication. FIG. 6, lower right panel, shows the release of the Dicamba-Gentian Violet, intense blue violet color when exposed to an ultrasonic bath for 5 minutes as compared to the non-sonicated keratin nanoparticle which has a lighter violet color.

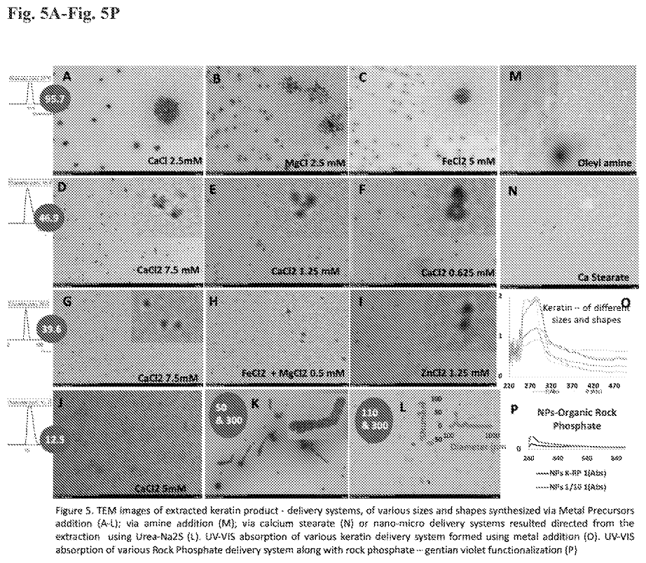

The natural extracts containing bio-polymers can be used directly in agricultural formulations as a biostimulant or engineered bio-polymers can be prepared by attaching one or more functional molecules to the extracted bio-polymers. The functional molecules include but are not limited to additional peptides, polymers (chitosan, gellan gum, cellulose, natural gum, rosin, paraffin, xanthan, alginate, agarose, polysaccharides, dextran, carrageenans, starch, albumin, polyacrylamide, polyester, polymethacrylate, polyamide, polyurethes, polycyanoacrylates, etc.), co-polymers, metal complexes, plant bioactive molecules, microelements (e.g. zinc, iron, manganese, copper, molybdenum, boron, chlorine or nickel), crosslinkers, or a combination thereof. The functional molecules are attached to the bio-polymer either covalently or non-covalently using linkers. The resulting engineered bio-polymer can have a chain shape (targeted bio-polymer), or they can self-assemble into nanoparticles (nano bio-polymer). Nano bio-polymers have an interior, exterior and an average diameter (hydrodynamic radius) between 1-1000 nm. Functional molecules can be embedded non-covalently with the bio-polymer and the resulting nano bio-polymer usually has a spherical shape. A functional linker can be added to an extracted bio-polymer dissolved in water or NADES. Bioactive agents can be added to an aqueous/or non-aqueous bio-polymer solution and the bioactive agent can be cross-linked or encapsulated on or in the bio-polymer or nanobio-polymer, and the bio-polymer and nano bio-polymer can act as a delivery system. See FIG. 5 which shows the TEM images of extracted keratin product delivery systems of various sizes and shapes synthesized via metal precursors (FIGS. 5A-L); via amine addition (FIG. 5M); via calcium stearate (FIG. 5N) or nano-micro delivery systems resulting from an extraction using Urea-Na2S (FIG. 5L). UV-VIS absorption of various delivery systems is shown in FIG. 5O.

Keratin-metal complexes of butterfly shape, dimer, trimers, wires or simple nanospheres can be synthesized using the present invention. In one embodiment using a metal precursor, langbernite and rock phosphate were used to obtain organic nanodelivery system composites. A change of color was observed in both cases, black grey solution for rock phosphate nanoparticles, and yellow for langbernite. The rock phosphate delivery systems can be obtaining by functionalizing them with an additional component. When the delivery system is used, the rock phosphate nanoparticles are exposed at a ratio of nanoparticles: additional component in a weight ratio of 1:10 and mixed for 1 h at room temperature and pH=7.4, then centrifuged at 5000 rpm for 20 min. FIG. 5P, shows the UV-Vis absorption spectra confirming both the formation of nanoparticles, having an absorption around 270 nm, (red line) and the formation of the delivery system with two absorption wavelengths, one corresponding to the nanoparticles at 274 nm, and one to gentian violet at 590 nm

The bio-polymers and nano bio-polymers can also have a gel or nanogel structure or they can be polymerized to enriched biopolymers by adding an additional component to form a waxy complex. The bio-polymers can be engineered using a dry or wet chemical reaction, pH modulation, precipitation or a combination thereof. The reaction can be initiated by addition of water 5-25% by weight ratio of the extract. A fatty compound can be added to produce a clay, wax enriched biopolymer, (around 1-15% fatty compound modification of biopolymer). Additional agricultural components can be added to develop specialized agricultural products. For example, an enriched biopolymer formulation can be synthesized for use as a seed priming or seed coating composition (see Example 11).

Nanoparticles, and nanodelivery systems can be formed by precipitation with a solvent, Tris buffers of various concentrations, such as 1 mM, 3 mM, 5 mM, 10 mM, and 20 mM; preferably 10 mM; or acetone, methanol, ethanol, or water, at various pHs can be used depending on the desired size of the nanoparticles. Proteins close to the isoelectric point will result in smaller nanoparticles. The increase elecronegativity of a pH around 7.3 to 9 results in bigger nanoparticles. Increasing the elecronegativity to a corresponding pH of 10 or above, causes the nanoparticles to collapse. Different shapes can be obtained by modulating the pH and the electronegativity at which the precipitation is carried out (see FIGS. 3 and 5 and Example 12).

Other classes of bio-polymers or extracts can be extracted and manipulated using the present invention. The polysaccharide chitin was extracted from shrimp shells, engineered to different sizes, and encapsulated with active agricultural compounds for various applications (see FIGS. 6A-H which show TEM images of acetylated chitin encapsulated with different pesticides. The UV-vis absorption spectra confirms the encapsulation (See FIG. 6I-J).

There are two types of delivery systems commonly used with agricultural formulations (1) systemic delivery systems which transport the encapsulated active compound into the plant and release it inside the plant; and (2) contact delivery systems which break when they contact the plant leaves and release the active compound. Systemic delivery systems can take longer to act, but they can be more effective because they work throughout the plant. Contact delivery systems can produce an immediate effect, but the effect does not last long. Each delivery system has pros and cons, and a customized delivery system formulation designed for specific applications could be used to control the delivery of the bio-polymer formulations and lower the indirect impact of the formulation on biota, humans, plants, soil and the environment. In the present invention, the delivery system can be customized by controlling the surface chemistry.

Additional functionalities can be incorporated into the delivery systems, for example, anchoring or targeting molecules can be attached to the surface of the delivery system. The delivery system can be a chain bio-polymer or a nano bio-polymer. In a preferred embodiment, a peptide anchoring molecule which acts as an adhesion promoter to hydrophobic surfaces and the waxy plant surface can be attached to the surface of the bio-polymer chains or nano bio-polymers for a contact delivery system. Enhanced adherence to the plant results in improved internalization, as well as controlled release of the chemical and intracellular targeted delivery. The resulting agricultural formulation can be applied to crops in any type of atmospheric conditions. The adhesion promotor results in adherence to the leaf surface, where the contact delivery system controls the release of the encapsulated active compound. This combines the benefit of an immediate effect with time-controlled release and reduces reapplication. Moreover, since the bio-polymers or nano bio-polymers can adhere to the leaves, the formulation offers protection against leaching off due to rains, avoids volatilization, reduces possible biota negative effects, and reduces plant and target overexposure. When extended or sustained release is desired after application, the trigger for release can include but is not limited to a pH change, a temperature change, a barometric pressure change, an osmotic pressure change, exposure to water, exposure to a solvent, changes in shear forces, exposure to a bacterium or exposure to an enzyme. In addition, a nanoparticle can be used which has a pore network and releases the encapsulated bio-polymers by diffusion.

Using a delivery system, the nutrient input (nitrogen or phosphorous) in crop production can be precisely controlled which will reduce the impact of the fertilizer on the health of the soil, ecosystem and people. In many state of the art compositions losses of up to 80% of the total agro-chemical quantity occur per application. Delivery systems can decrease the high losses of chemicals, reduce the over-application and the run-off of the active toxic chemicals. Based on their nanoscale properties, formulations containing nano bio-polymers will be more potent, will require lower application doses, can be used in lower concentrations, and will improve productivity as compared to conventional formulations. Plant physiology—growth stimulation can be controlled directly by internalization of nano bio-polymers into various cellular compartments, or plant physiology—growth stimulation can be influenced by targeting the soil microbiota to enhance their communication with the plant for plant growth, development, and protection. In addition to peptide anchoring molecules which act as adhesion promotors, bioactive molecules, pH sensitive molecules, thermo-responsive molecules, or a combination thereof can be linked to the bio-polymers or nano bio-polymers resulting in bio-polymers and nano bio-polymers having specific responsive properties which can be used as smart targeted delivery systems.

The bioactive molecules can be linked directly to the initial bio-polymer, and the initial bio-polymer can be used as a targeted delivery system. In another embodiment, the targeted delivery system can include functional polymers which have bioactive molecules linked to a polymer or co-polymer. The functional polymers are attached to the initial bio-polymer to form the engineered bio-polymer and nanobio-polymer. The functional polymers can be a single molecule sequence which is attached to the surface of the bio-polymer or nanobio-polymer. The functional molecules and the initial bio-polymerscan both act as bioactive molecules. Bioactive molecules can also be encapsulated within the nanobio-polymers, bio-polymer gels or nano bio-polymer gels, and their surface can be further functionalized by attaching additional targeting molecules, anchor molecules, or combinations thereof.

The bio-polymers and nano bio-polymerscan be formulated to be pH responsive by selecting different sources of peptides with strategic isoelectric points. In addition, pH sensitive molecules can be attached to the bio-polymers and nanobio-polymers. The pH sensitive molecule can be a peptide, or a polymer, co-polymer or a combination thereof that responds to changes in the pH of the environment. When the pH of the surrounding medium changes to the designated pH, the delivery system will release the bioactive compounds immediately or at a controlled rate over a period of time. In addition to changes in pH, the trigger for release can be a temperature change (thermo-responsive), barometric pressure change, osmotic pressure change, exposure to water, exposure to a solvent, changes in shear forces, application of the formulation, exposure to a bacteria, exposure to an enzyme, exposure to electromagnetic radiation and exposure to free radicals, depending on the functional molecules which are attached to the bio-polymer and nanobio-polymer. The release of the bioactive molecules can also be controlled by using nano bio-polymers with a specific structure such as a cavity or a network structure to encapsulate the bioactive molecule.

The bio-polymer and nano bio=polymer can be combined with other beneficial agents including but not limited to plant growth regulators, bactericides, fungicides, insecticides, acaricides, miticides, nemanticides, molluscicides, herbicides, plant nutrients (e.g. potassium, phosphate, carbohydrates, and amino acids), preservatives, microelements, humectants, detergents, fertilizers, chelating compounds, organic acids, surfactants, dispersing agents or combinations thereof.

Formulations containing bio-polymers and/or nano bio-polymers can be used as herbicides. The protein:herbicide can be prepared at a weight ratio 1:3; 1:6:1:6.6; 1:10:1:16.6; 1:30 or 1:60. The formulation can be made to correspond to 2.25 kg Dicamba application per Ha, or can correspond to a lower or higher concentration. Additionally, the nanoparticle-herbicide delivery system can be preserved for up to 1 year. A DES ChCl: Glycerol, can be prepared at a different molar ratio, such as 1:1; 1:2; 1:3; 1:4, preferably 1:2, and preferably between 1-3% of glycerol in the final diluted application formulation. No preservatives or softeners or anti-volatile compounds are necessary. The herbicide delivery systems can be added to various agricultural formulations, or can be combined with other herbicides that are known in the art.

Formulations containing bio-polymers and/or nano bio-polymers can be used as fertilizers. A multipurpose fertilizer can be prepared using 5-30% extracted bio-polymers, nano bio-polymers or a combination thereof. The bio-polymers can be extracted from any bio-source and the nano bio-polymers can be formed by a crosslinking reaction or precipitation. The fertilizer base formulation contains 5-30% extracted bio-polymers and/or nano bio-polymers, 10-30% nitrogen, 2-20% phosphorus, 2-20% potassium, 2-8% chelated microelements or nanoelements, 10-30% amino acids and 1-3% carbohydrates. The initial bio-polymerscan contain one or more chelated metals. A fertilizer composition for metal deficient crops can include 3-30% bio-polymers chelate formed with 0.1-3% metal ions selected from iron, zinc, manganese, and copper and/or 3-30% nano bio-polymers with 0.1-3% metal ion nanoparticles (e.g. iron nanoparticles, zinc nanoparticle, manganese nanoparticles, and copper nanoparticles) encapsulated therein. Anchoring molecules such as anti-microbial peptides are preferably attached to the extracted bio-polymers or nano bio-polymers.

Extracted bio-polymers and nano bio-polymers have a high impact on plant physiology. In addition to acting as a biostimulant, extracted bio-polymers and nano bio-polymers can also alleviate the negative effects of abiotic plant stress due to salinity, drought and heavy metals. Extracted bio-polymers and nano bio-polymers directly affect plants by stimulating carbon and nitrogen metabolism and interfering with hormonal activity. Indirectly, bio-polymers and nano bio-polymers could enhance nutrient availability in plant growth substrates and increase nutrient uptake and nutrient-use efficiency in plants. Bio-polymer and nano bio-polymer delivery systems that carry a bioactive agent act as both a bio-stimulant based on their polymer composition, and as a stimulant based on the function of the bioactive agent. For example, when the bioactive agent is selected from a class that targets a pathogen the bio-polymer delivery system acts both as a biostimulant to stimulate plant growth and the immune system, and as an anti-pathogen to treat and/or protect the plant from various diseases. The bio-polymers and nano bio-polymers are designed to have a specific size, surface charge and conformation. Functional groups can be attached to the initial bio-polymer structure to provide specific characteristics such as mechanical stability, pH or temperature responsive properties, or specific denaturation properties.

### Extraction of Initial Bio-Polymers

A diversity of products can be extracted using the process of the present invention. The DES/NADES of the present invention can also be used to dissolve a product or used in a formulation to preserve a product or a combination of products. The extracted or dissolved material can include but are not limited to a protein, a DNA, a RNA, a polysaccharide, an enzyme, a toxin, a flavonoid, an anthocyanin, a colorant, an alkaloid, a terpenoid, a phenylpropanoid a glycoside, a phenolic compound, a colorant, a flavor, a fragrance, a dye, an herbicide, an insecticide, a pesticide, a biostimulant to the plant, a biocide or a mixture thereof. The resulting products can be used in many applications including but not limited to agriculture, food, and cosmetics.

The initial bio-polymerscan be extracted from any organic materials, preferably the initial bio-polymers are extracted from bio-waste materials. The bio-waste sources can be animal in origin and can include any animal by products including but not limited to animal epithelial or connective tissues, blood meal, fish tissue, feathers, hair, bones, skin, shellfish shells, wool waste from clothing manufacturing and leather by-products. The bio-waste sources can be plant in origin, including but not limited to crop waste (corn stalks, sugarcane bagasse, prunings and culls), food processing by-products (corn cobs, chaff, straw, pomace), brewery waste (spent grain) and wood.

The extraction of the biomass using NADES can be tailored by choosing different types of NADES/DES, with specific acidity, basicity, composition and molar ratios, to control the molecular weight, size, purity, and structure to obtain nanosized, microsized, nanowired and nanoparticles of different shapes and sizes. They can also be combined with methods such as a dry chemistry method, a wet chemistry method or metal oxide methods, for reducing the size of the bio-polymers, and to encapsulate and/or surface functionalize the resulting products. Additionally, the extraction can be combined with methods such as assisted ball milling or ultrasonication. By combining NADES with ball milling and ultrasonication, novel bioactive components can be easily functionalized to the bio-polymer surface or encapsulated inside or between their structure or all of the above, and the resulting compound has novel properties. Further, ball milling and sonication reduce the extraction time, increase the efficiency of extraction, and lowers the costs.

After extraction of the biomass, the NADES can be recycled or can be left in the agricultural formulation. NADES does not have adverse effects on humans, plants, or the environment and in some cases, the NADES can act as a bio stimulant to the plant. NADES can achieve a biomass extraction yield between 85-98%, preferably 92-98%, and purity between 80-100%, preferably 95.3 to 100%.

The initial bio-polymer can be engineered to carry one or more bioactive compounds, and release them in soil/hydroponic media or on plant leaves, or to deliver them inside the plant, all in a controlled, targeted and timely manner. The engineered bio-polymer, can be used as a smart delivery system in agricultural formulations. The smart delivery system can minimize leaching while improving the uptake of nutrients by plants and can mitigate eutrophication by reducing the transfer of the bioactive compound to the ground. More than 85% of conventional fertilizers and pesticides are leaching into rivers, degrading and volatilizing. Leaching, volatilizing and degrading can be avoided or reduced by encapsulating agricultural formulations into nanoparticles or gels. The chemicals are internalized into the plant inside the nanoparticle and/or on the surface of a nano bio-polymer or bio-polymer. Moreover, the nano bio-polymer allows improved delivery, targeted accumulations and controlled releases of the attached or encapsulated agricultural agent into the plant.

The structure and function of the encapsulated/attached bioactive compounds are protected by the bio-polymer, which provides increased solubility and resistance against hydrolysis and photodecomposition. For example, it has been reported that 75-95% of phosphorous fertilizers form complexes and/or precipitates in the soil, and only 5-25% is absorbed by the plant. The delivery systems of the invention efficiently deliver a phosphate precursor nutrient resulting in greater phosphate efficiency and lower losses. Moreover, herbicides and pesticides are well known for their toxic effects both on the environment and humans. Encapsulating pesticides and herbicides will result in a lower volatilization loss, thus protecting the environment and human health. In addition, lower dosages of encapsulated chemicals are needed to provide the desired effect. Since the bio-polymers are bioactive compounds (i.e. act as nutrients), when applied in combination with pesticides and/or herbicides, the formulation provides both protection and stimulates plant growth. The formulations can be applied as a component of a seed coating blend. Seed treatment formulations with extracted bio-polymers and nano bio-polymers mixed with antimicrobials, fungicides and insecticides can reduce later pesticide applications.

Bio-waste means a raw material which consists primarily of the organic residue from processed materials. Bio-waste can include but is not limited to (1) animal by-products left after processing of meat and seafood (bones, tendons, skin, the contents of the gastro-intestinal tract, blood and internal organs, feet, feathers, shells, hair, etc.), waste from the garment industry (hide, wool, etc.); (2) plant by-products including but not limited to crop residue, plant parts leftover from the production of food, weeds, leaf litter, sawdust, forest waste, paper mill sludge, and livestock waste (manure); (3) yeast by-products including but not limited to waste yeast produced by fermentation, and (4) municipal waste.

Extracted bio-polymers encompass at least one peptide, lipid or polysaccharide that was extracted from bio-waste; a combination of peptides, lipids and/or polysaccharides extracted from bio-waste, cross-linked peptides, lipids and/or polysaccharides and engineered extracted peptides, lipids and/or polysaccharides, or a combination thereof.

The term peptide refers to a compound comprised of amino acid residues. A peptide must contain at least two amino acids, but no limitation is placed on the maximum number of amino acids that can comprise a peptide sequence. As used herein, the term refers to both short chains commonly referred to in the art as peptides, oligopeptides and oligomers, and to longer chains, which generally are referred to in the art as proteins.

An engineered bio-polymer has at least one functional group attached to its surface.

A functional group, is any molecule which changes the properties of the bio-polymer such as a bioactive molecule, an additional polymer, a co-polymer, a linker, a targeting molecule, an anchoring molecule, a pH responsive molecule, a thermosensitive molecule and combinations thereof.

A gel is a semi-solid nonfluid polymer which has a dilute cross-linked network within a fluid.

A nanogel is a nanoparticle composed of a hydrogel containing a highly cross-linked polymer network. Nano gels are 3-1000 nm in size.

A pH sensitive or pH responsive molecule responds to changes in the pH of the surrounding environment by changing properties such as size or shape.

A linker is an intermediary short molecule for bonding the initial bio-polymer and another functional molecule through covalent bonding. It can attach to specific groups by using one or more active groups. Linker precursors for the engineering of bio-polymers and nano bio-polymers can be selected from but are not limited to different classes of fatty amines or amido (fatty amines/amidoaines are compounds that contain amines/amidoamines and are hydrophobic), or/and metal oxides or/and metal chlorides, different classes of fatty metals (a fatty metal is a metal that is attached to a hydrophobic molecule), a polymer from any class of polymers, a DNA, a protein, a RNA, a N-(α-Maleimidoacetoxy)-succinimide ester, N-5-Azido-2-nitrobenzyloxy-succinimide, 1,4-Bis-Maleimidobutane, 1,4-Bis-Maleimmidyl-2,3-dihydroxy-butane, Bis-Maleimidohexane, Bis-Maleimidoethane, N-(β-Maleimidopropionic acid)hydrazide, N-(β-Maleimidopropyloxy)succinimide ester, 1,8-Bis-Maleimidodiethylene-glycol, 1,11-Bis-Maleimidotriethyleneglycol, (Sulfo-DSS) Bis (sulfosuccinimidyl)suberate, Bis (sulfosuccinimidyl)glutarate-d, Bis(sulfosuccinimidyl)2,2,4,4-glutarate-d, Bis (sulfosuccinimidyl)suberate-d, Bis (sulfosuccinimidyl)2,2,7,7-suberate-d, BS(PEG)5 Bis (NHS)PEG5, BS(PEG)9 Bis (NHS)PEG, Bis (2-[succinimidoxycarbonyloxy]ethyl)sulfone, DCC N,N-Dicyclohexylcarbodiimide, 1-5-Difluoro-2,4-dinitrobenzene, Dimethyl adipimidate·2HCl, DMP Dimethyl pimelimidate·2HCl, Dimethyl suberimidate·2HCl, Disuccinimidyl glutarate, Dithiobis(succimidylpropionate) (Lomant's Reagent), Disuccinimidyl suberate, Disuccinimidyl tartarate, Dimethyl 3,3′-dithiobispropionimidate·2HCl, Dithiobis-maleimidoethane, (Sulfo-DSP) 3,3′-Dithiobis (sulfosuccinimidylpropionate), EDC 1-Ethyl-3-(3-dimethylaminopropyl) carbodiimide, Ethylene glycol bis (succinimidylsuccinate), N-ε-Maleimidocaproic acid, N-(ε-Maleimidocaproic acid)hydrazide, N-(ε-Maleimidocaproyloxy)succinimide ester, N-(γ-Maleimidobutyryloxy)succinimide ester, N-(κ-Maleimidoundecanoic acid)hydrazide, NHS-LC-Diazirine, Succinimidyl 4-(N-maleimidomethyl), cyclohexane-1-carboxy-(6-amidocaproate), Succinimidyl 6-(3′-[2-pyridyldithio]propionamido)hexanoate, L-Photo-Leucine, L-Photo-Methionine, m-Maleimidobenzoyl-N-hydroxysuccinimide ester, 4-(4-N-Maleimidophenyl)-butyric acid hydrazide·HCl, 2-[N2-(4-Azido-2,3,5,6-tetrafluorobenzoyl)-N6-(6-biotinamidocaproyl)-L-lysinyl]ethylmethanethiosulfate, 2-{N2-[N6-(4-Azido-2,3,5,6-tetrafluorobenzoyl)-N6-(6-biotinamidocaproyl)-L-lysinyl]}ethylmethanethiosulfate, NHS N-Hydroxysuccinimide, NHS-Azide N-hydroxysuccinimide ester ethane azide, NHS-PEG4-Azide N-hydroxysuccinimide ester tetraoxapentadecane azide, N-hydroxysuccinimide ester dodecaoxanonatriacontane azid, 3-(2-Pyridyldithio)propionylhydrazide, 2-pyridyldithiol-tetraoxatetradecane-N-hydroxysuccinimide, 2-pyridyldithiol-tetraoxaoctatriacontane-N-hydroxysuccinimide, N-(p-Maleimidophenyl)isocyanate, Succinimdyl 3-(bromoacetamido)propionate, NHS-Diazirine, NHS-SS-Diazirine, N-succinimidyl iodoacetate, N-Succinimidyl(4-iodoacetyl)aminobenzoate, Succinimidyl 4-(N-maleimido-methyl)cyclohexane-1-carboxylate, NHS-PEG2-Maliemide, NHS-PEG4-Maliemide, NHS-PEG6-Maleimide, NHS-PEG8-Maliemide, NHS-PEG12-Maliemide, NHS-PEG24-Maleimide, Succinimidyl 4-(p-maleimido-phenyl)butyrate, Succinimidyl-6-(β-maleimidopropionamido)hexanoate, 4-Succinimidyloxycarbonyl-methyl-α-(2-pyridyldithio)toluene, Succinimidyl-(4-psoralen-8-yloxy)butyrate, N-Succinimidyl 3-(2-pyridyldithio)propionate, Sulfo-EGS Ethylene glycol bis (sulfo-succinimidyl succinate), N-(ε-Maleimidocaproyloxy)sulfosuccinimide ester, N-(γ-Maleimidobutryloxy)sulfosuccinimide ester, N-(κ-Maleimidoundecanoyloxy)sulfosuccinimide ester, Sulfo-NHS-LC-Diazirine, Sulfosuccinimidyl 6-(3′-[2-pyridyldithio]propionamido)hexanoate, m-Maleimidobenzoyl-N-hydroxysulfosuccinimide ester, Sulfo-NHS N-Hydroxysuccinimide, Sulfo-NHS-Phosphine, Sulfosuccinimidyl 6-(4′-azido-2′-nitrophenylamino)hexanoate, Sulfo-NHS-(2-6-[Biotinamido]-2-(p-azidobezamido), Phenyl azide, Biotin, Sulfo-NHS-Diazirine, Sulfo-NHS-SS-Diazirine, Sulfosuccinimidyl(4-iodo-acetyl)aminobenzoate, Sulfosuccinimidyl 4-(N-maleimidomethyl)cyclohexane-1-carboxylate, Sulfosuccinimidyl 4-(p-maleimidophenyl)butyrate, Tris-(2-Maleimidoethyl)amine (Trifunctional), and Tris-(succimimidyl aminotricetate) (Trifunctional).

Targeted bio-polymers refers to specific classes of bio-polymers that are structurally targeted towards a specific application. They are tailored to meet specific requirements for degradation, bioactive molecule release or incorporation of different hydrophobic or hydrophilic tails. Targeted bio-polymers can be used to control surface charge, pH and stimuli responses, denaturation and targeted delivery to a specific site. Anchoring or internalization peptides can be attached to the bio-polymers surface. Anchoring peptides such as antimitotic peptides enhance their adherence to plant leaves. Cell penetrating peptides (CPPs) can enhance the transport of the bio-polymers into cells and specific cell compartments.

Anchoring peptides refer to a specific class of peptides which in addition to their defense-related properties (e.g. antibacterial, antifungal, antiviral, anti-oxidative activity, and chitinase and proteinase inhibitory activities), display “peptide promiscuity”, which refers to multiple functions displayed by a single peptide. The anchoring peptides have the ability to increase the attachment to the surface of the waxy plant leaves, which allows the agricultural formulation to be applied in any conditions, including rain, by reducing run off of the formulation. These classes of peptides have a sequence less than 100 amino acids, preferably between 10-100 amino acids, and contain specific variations of amino acid constituents. These peptides have a MW of 2-125 kDa, preferably 2-6 kDa. In addition, their amino acid structure is compact with high thermal, chemical, and enzymatic anchoring properties. The anchoring peptides have specific secondary and/or tertiary structures and contain two or three domains: N- and C-terminal pro-domains or N- and C-terminal pro-domains and a third domain which can be a mature function related peptide domain. The third domain has an antimicrobial, antifungal, antiviral, and/or anti-oxidative activity, chitinase and/or proteinase inhibitory activities, anchoring activity or a combination thereafter.

Anchoring peptides have the ability, when applied on plants, to organize into specific structures with conserved structural folds that enable sequence variation of amino acid residues that are encased in the same scaffold within a particular structure. The anchoring peptides include but are not limited to peptides from families including thionins, defensins, hevein-like peptides, knottin-type peptides (linear and cyclic), lipid transfer proteins, α-hairpinin families, snakins, Glycine-rich peptide, His-rich peptide shepherins, eGFP-anchor-peptide fusion protein, Plantaricin A (22 aa), Cn-AMP1 and Cr-ACP1.

Polymer precursors for the engineering of the bio-polymers and nano bio-polymers are biodegradable polymers including but not limited to polyethylene glycol (PEG), polyglycolic acid (PGA), polylactic acid (PLA), lactic acid-glycolic acid copolymer (PLGA), polyhydroxyalkanoates (PHA), polyhydroxybutyrate-valerate (PHBV), polyvinyl alcohol (PVA), polyethylene terephthalate (PET), polyglycolide-lactide, polycaprolactone (PCL), lactic acid-s-caprolactone copolymer (PLCL), polydioxanone (PDO), polytrimethylene carbonate (PTMC), poly(amino acid), polydioxanone, polyoxalate, a polyanhydride, a poly(phosphoester), polyorthoester, poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyhydroxyalkanaates, polyphosphoester, polyphosphoester urethane, poly(amino acids), cyanoacrylates, poly(trimethylene carbonate), poly(iminocarbonate), copoly(ether-esters) (e.g., PEO PLA), polyalkylene oxalates, polyphosphazenes. Cross-linked acrylic polymers, polypropylene, polyurethane, polyurethane foams, mixtures thereof, copolymers thereof and combinations thereof.

Nano bio-polymers are any nanoscale delivery system which includes at least one bio-polymer or a combination of bio-polymers and a biodegradable polymer or a copolymer. Nano bio-polymers are synthesized using crosslinkers, added directly to the extracted bio-polymer solution. They are generated having a specified size without the need for any further purification and have a high loading capacity of bioactive agents. Nano bio-polymers produce high internalizations in plants. Additionally, they can be formulated to be responsive to pH and/or temperature or their surface can be modified with anchoring or targeting molecules. Nano bio-polymers with specific properties can be made by selecting specific classes of bio-polymers and modifying their physical-chemical properties, their log P and/or by incorporating anchoring molecules, targeting agents and/or internalization molecules. The nano bio-polymer composition may be formulated for foliar application to penetrate the stomatal pores of the plant leaves and/or the nano bio-polymer composition can be applied on the soil and taken up through the soil by the root systems in plants. Nano bio-polymers have a particle size of from about 3 nm to about 1000 nm, preferably from about 3 nm to about 500 nm, more preferably from about 3 nm to about 300 nm, and most preferably from about 3 nm to about 100 nm. When using nano bio-polymers with a size greater than 100 nm, in order to avoid the possibility of toxicity to humans by inhalation, bulk peptides can be agglomerated to microscale using different stimuli and pHs.

Nanoparticle refers to any particle having an average diameter between 3 nm to 1000 nanometers (nm). In some embodiments, nanoparticles have an average diameter of less than 500 nm, less than 300 nm, less than 200 nm, less than 100 nm, less than 50 nm, less than 25 nm, less than 10 nm or less than 5 nm. A nanoparticle formulation or composition means any substance in a liquid or solid form that contains at least one nanoparticle. In some embodiments, a nanoparticle formulation is a uniform collection of nanoparticles.

The term copolymer means a polymer formed from two or more polymers. By way of example and without limitation, a copolymer can be an alternating copolymer, a random copolymer, a block copolymer, or a graft copolymer.

Bactericide and bactericidal refers to a substance or mixture of substances that kill or inhibit the growth of a bacterium that is pathogenic to plants or animals. Exemplary bactericides include, but are not limited to bronopol; chlorothalonil+copper+maneb, chloropicrin; dichlorophen; fosetyl-AL; nitrapyrin; nickel dimethyldithiocarbamate; kasugamycin; octhilinone; furancarboxylic acid; oxytetracycline; probenazole; sodium hypochlorite; streptomycins; tecloftalam; and copper compounds, such as copper oxychloride, copper hydroxide and copper sulfate.

Antimicrobial and antimicrobial agent refer to a substance or mixture of substances that kills or inhibits the growth of a microorganism that is pathogenic to plants or animals; such as a bacterium, fungus or virus.

Fungicide and fungicidal agent refer to a substance or mixture of substances that kill or inhibit the growth of a fungus that is pathogenic to plants or animals.

Pesticide and pesticidal agent refer to a substance or mixture of substances that kill or inhibit the growth of a plant pest that is pathogenic to plants, including insecticides, acaricides, miticides, nemanticides and molluscicides.

Insecticide and insecticidal agent refer to a substance or mixture of substances that kill or inhibit the growth of an insect that is pathogenic to plants. As used herein, the term “insects” includes all organisms in the class “Insecta” and encompasses “pre-adult” insects, which include any form of an organism prior to the adult stage, including, for example, eggs, larvae, and nymphs.

Acaricide and acaricidal agent refer to a substance or mixture of substances that kill or inhibit the growth of a member of the arachnid subclass Acari that is pathogenic to plants or crops, which includes ticks and mites.

Miticide and miticidal agent refer to a substance or mixture of substances that kill or inhibit the growth of a mite that is pathogenic to plants or crops.

Nematicide and nematicidal agent refer to a substance or mixture of substances that kill or inhibit the growth of a nematode that is pathogenic to plants or crops. The term nematode comprises eggs, larvae, juvenile and mature forms of nematodes. Exemplary nematicides include but are not limited to etridiazole, spiroxamine, fluopicolide, phosphorous acid, triadimefon+trifloxystrobin, and combinations thereof.

Molluscicide and molluscidal agent refer to a substance or mixture of substances that kill or inhibit the growth of a mollusc, such as a gastropod pest (e.g., slugs and snails) that is pathogenic to plants. Exemplary molluscicides include but are not limited to metal salts, such as iron (iii) phosphate and aluminum sulfate, metaldehyde, methiocarb, acetylcholinesterase inhibitors, and combinations thereof.

Herbicide and herbicidal agent refer to a substance or mixture of substances that selectively kill or inhibit the growth of unwanted plants. Exemplary herbicides include but are not limited to Anilides, such as Ditlufenican and Propanil; Arylcarboxylic acids, such as Dichloropicolinic acid, Dicamba and Picloram; Aryloxyalkanoic acids, such as 2,4-D, 2,4-DB, 2,4-DP, Fluroxypyr, MCPA, MCPP and Triclopyr, Aryloxy-phenoxy-alkanoic esters, such as Diclofop-methyl, Fenoxaprop-ethyl, Fluazifop-butyl, Haloxyfop-methyl and Quizalotop-ethyl; Azinones, such as Chloridazon and Norflurazon; Carbamates, such as Chlorpropham, Desmedipham, Phenmedipham and Propham; Chloroacetanilides, such as Alachlor, Acetochlor. Butachlor, Metazachlor, Metolachlor, Pretilachlor and Propachlor; Dinitroanilines, such as Oryzalin, Pendimethalin and Trifluralin; Diphenyl Ethers, such as Acitluorfen, Bifenox, Fluoroglycofen, Fomesafen, Halosafen, Lactoten and Oxyfluorfen; Ureas, such as Chlortoluron, Diuron, Fluometuron, Isoproturon, Linuron and Methabenzthiazuron; Hydroxylamines, such as Alloxydim, Clethodim, Cycloxydim, Sethoxydim and Tralkoxydim; Imidazolinones, such as Imazethapyr, Imazamethabenz, Imazapyr and Imazaquin; Nitriles, such as Bromxynil, Dichlobenil and loxynil; Oxyacetamnides, such as Mefenacet; Sulfonylureas, such as Amidosulfuron. Bensulfuron-methyl, Chlorimuron-ethyl, Chlorsulfuron, Cinosulfuron, Metsulfuron-methyl, Nicosulfuron, Primisulfiuron, Pyrazosulfuron-ethyl. Thifensulfuron-methyl, Triasulfuron and Tribenuron-methyl; Thiolcarbamates, such as Butylate, Cycloate, Diallate, EPTC, Esprocarb, Molinate, Prosulfocarb, Thiobencarb and Triallate; Triazines, such as Atrazine, Cyanazine, Simazine, Simetryne, Terbutryne and Terbutylazin; triazinones, such as Hexazinone, Metamitron and Metribuzin; and others, such as Aminotriazole, Beefuresate, Bentazon, Cinmethylin, Clomazone, Clopyralid, Ditenzoquat, Dithiopyr, Ethofumesate, Fluorochloridone, Gibberellic acid, Glufosinate, Glyphosate, Isoxaben, Pyridate, Quinchlorac, Quinmerac, Sulphosate, Tridiphane, Dalapon, Glyphosine, loxynil, Chlorfluorenol, Dichlorprop, Dichlofop, Mecoprop, Chlormequat, Diquat, Paraquat, Chloroacetic acid, Fluazifop, Pyridate, Chlorsulfuron, Flurenol, Sulfometuron, and natural oils.

Plant growth regulator refers to a substance or mixture of substances which accelerate or retard the rate of growth or maturation or otherwise alter the behavior of seeds, plants, or the produce thereof (e.g., seed germination, root growth, development processes, plant growth, maturation, and senescence, fruit set, and fruit drop) through physiological action(s). Plant growth regulators do not include substances or mixtures of substances substantially serving as plant nutrients, micronutrients, nutritional chemicals, plant innoculants, desiccants, biocides, pesticides, herbicides, or soil amendments. Exemplary plant growth regulators include but are not limited to antiauxins, auxins, cytokinins, defoliants, ethylene inhibitors, ethylene releasers, gametocides, gibberellins, growth inhibitors, growth retardants, growth stimulators, and unclassified growth regulators. Exemplary antiauxins include, but are not limited to clofibric acid and 2,3,5-tri-iodobenzoic acid. Exemplary auxins include, but are not limited to, 4-CPA, 2,4-D, 2,4-DB, 2,4-DEP, dichlorprop, fenoprop, IAA, IB A, naphthalene acetamide, a-naphthalene acetic acids, 1-naphthol, naphthoxy acetic acids, potassium naphthenate, sodium naphthenate, and 2,4,5-T. Exemplary cytokinins include, but are not limited to, 2iP, benzyladenine, 4-hydroxyphenethyl alcohol, kinetin, and zeatin. Exemplary defoliants include, but are not limited to, calcium cyanamide, dimethipin, endothal, ethephon, merphos, metoxuron, pentachlorophenol, thidiazuron, and tribufos. Exemplary ethylene inhibitors include, but are not limited to, aviglycine and 1-methylcyclopropene.

Exemplary ethylene releasers include, but are not limited to, ACC, etacelasil, ethephon, and glyoxime. Exemplary gametocides include, but are not limited to, fenridazon and maleic hydrazide. Exemplary gibberellins include, but are not limited to, gibberellins and gibberellic acid. Exemplary growth inhibitors include, but are not limited to abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat, piproctanyl, prohydrojasmon, propham, tiaojiean, 2,3,5-tri-iodobenzoic acid and morphactins, such as chlorfluren, chlorflurenol, dichlorflurenol and flurenol. Exemplary growth retardants include, but are not limited to, chlormequat, daminozide, flurprimidol, mefluidide, paclobutrazol, tetcyclacis, uniconazole. Exemplary growth stimulators include, but are not limited to, brassinolide, brassinolide-ethyl, DCPTA, forchlorfenuron, gamma-aminobutyric acid, hymexazol, prosuler, pyripropanol and triacontanol. Exemplary signaling agents include, but are not limited to, Ca²⁺, inositol phospholipids, G-proteins, cyclic nucleotides, protein kinases, protein phosphatases and sodium glutamate. Exemplary unclassified plant growth regulators include, but are not limited to, bachmedesh, benzofiuor, buminafos, carvone, choline chloride, ciobutide, clofencet, cloxyfonac, cyanamide, cyclanilide, cycloheximide, cyprosulf amide, epocholeone, ethychlozate, ethylene, fuphenthiourea, furalane, heptopargil, holosulf, inabenfide, karetazan, lead arsenate, methasulfocarb, prohexadione, pydanon, sintofen, Iriapenthenol and trinexapac.

Abiotic stress refers to nonliving environmental factors, such as frost, drought, excessive heat, high winds, etc., that can have harmful effects on plants

Bioactive molecules provide a local or systemic biological, physiological or therapeutic effect in the biological system to which it is applied. Bioactive molecules used in agriculture include but are not limited to bio-polymers including hydrolyzed peptides, nano hydrolyzed peptides, polysaccharides and lipids, bactericides, fungicides, insecticides, acaricides, miticides, nematicides, molluscicides, herbicides, plant nutrients, plant growth regulators, nucleic acid sequences, compounds containing nucleic acids, microbes, peptide agents, and microelements. Other bioactive agents include one or more substances that may enhance defense mechanisms of the plant, including but not limited to acibenzolar-S-methyl, azadirachtin, phosphorous acid or phosphite salts, and the like.

As used herein, the terms plant nutrient material, plant nutrient and fertilizer refer to any material, elements, compounds or compositions that can be used as nutrient for a plant. Examples of plant nutrient material include but are not limited to, nitrogen fertilizer materials, such as anhydrous ammonia, urea, ammonium nitrate, ammonium sulfate; phosphorus fertilizer materials such as diammonium phosphate, monoammonium phosphate, triple superphosphate, ordinary superphosphate, ammonium polyphosphate; potassium fertilizer materials such as potassium chloride, potassium sulfate, potassium nitrate; secondary nutrients and micronutrients such as magnesium, magnesium oxy-sulfate (granular), dolomitic limestone, magnesium sulfate (Epsom salts), magnesium-potassium sulfate, sulfur, K-Mag (Sul-po-mag), calcium sulfate (Gypsum), ammonium sultate, boron, borax, solubor, calcium, calcitic limestone, bone meal, iron, iron sulfate, iron chelates, manganese, manganese oxy-sulfate, manganese chelates (soluble powder), zinc, zinc oxy-sulfate and zinc chelates.

### Extraction and Ultrasonication

Keratin can be extracted from wool and hair, collagen from shrimp and fish, thionin from plant leaves and stems, cellulose from wood and inorganic components including but not limited to flavonoids and phenols, can be extracted by using ChCl-oxalic acid as the DES solvent. Different carbohydrates, dietary fiber, sugars fat, protein, vitamins, and acids can be extracted using the same method but changing the reaction conditions, NADES type, time, and/or temperature.

For instance, in order to extract collagen with an efficiency of 92.5% to 98%, and a purity of 95.3% the reaction time is between 2-4 hours at a temperature between 65-80° C. The pH is adjusted to 4.5-7.0. The solvent: solid ratio is between 80:1 to 200:1, preferably 170:1, and can be modulated to result in low or high molecular weight peptides. The initial peptide and the chain peptides (PHs) can have a molecular weight from about 2 kDa to about 1000 kDa, preferably from 2 kDa to 500 kDa, more preferably 2 kDa to 50 kDa. The optimum extraction for high molecular weight peptides is ChCl-OA (1:1.0)/waste (80:1, mL/g) at 65° C. for 2 hours; for low molecular weight peptides the optimum extraction is ChCl-OA (1:1.0)/waste (120:1, mL/g) at 65° ° C. for 6 h. Ultrasound sonication can be carried out between 100 W to 800 W to produce nanoparticle delivery systems. The extraction and sonication can be performed in the same time.

In order to extract collagen with an efficiency of 62.5% to 93%, and a purity of 80-95.3%, bio-NADES can be prepared using waste materials from sugar production. 10 g of citric acid can be obtained from the fermentation of orange peel waste and mixed with 2.5 g of molasses waste (containing glucose, fructose and sucrose of 1.3 g) and 25% water. The mixture is stirred at 50° C., until a clear viscous liquid is formed (slurry bio-NADES). Optionally, the liquid can be separated to obtain pure bio-NADES. The slurry bio-NADES or the pure bio-NADES is mixed with ground fish skin waste at a of ratio 300: 0.5 mg/g, preferably 120:1 ml/g and the extraction reaction is carried out at 65° C. for 2 hours with a resulting extraction efficiency of 62.5%. To increase the extraction efficiency to 93% the reaction can be carried out for an additional 4 h.

Varying the Size of Extracted Bio-Polymers with Ball-Milling in DES or NADES

Extracted bio-polymers are hydrated in DES, NADES or bio-NADES (NADES made from bio waste) for between 10 min to 8 hours, at temperatures between 20-120° C. The hydrated bio-polymers are subjected to a mechanical process using a planetary ball milling at speeds between 100 to 800 rpm. Optionally, functional groups and/or bioactive compounds can be attached to the bio-polymers by adding the functional groups and/or bioactive agents during ball milling to produce functional—bio-polymers with attached or encapsulated bioactive agents.

The mechanical action of the ball milling on the extracted bio-polymers, combined with the chemical action of the DES or NADES produces various sizes and shapes of micro and nano bio-polymers with specific crystallinity and properties.

### Wet-Chemistry Crosslinking Using Covalent and Non-Covalent Functionalization

The initial extracted bio-polymer is engineered using a functionalization reaction, such as attaching at least one functional group or a combination of functional groups, at a specific pH, to the initial bio-polymer at targeted groups as shown in Table 3. Specific structures such as a chain like structure; a self-assembled nanoparticle structure; or their gels can be produced. Additional functionalization can be performed to attach distinct functional groups, or a combination of functional groups, either to amino acids of the initial peptide or to previously attached functional groups, or both.

In other words, a first functionalization is performed by adding the first selected functional groups or the combination of functional groups to the initial bio-polymer surface. Subsequently a second functionalization is performed by attaching the second selected functional groups, or a combination of functional groups to the engineered bio-polymer resulting from the first functionalization. A third functionalization is performed by attaching the third selected functional groups to the engineered bio-polymer of the second functionalization. Additional functionalization can be carried out.

Linkers can act as intermediates between the desired functional molecules and the targeted groups which form the extracted bio-polymer structure. The targeted groups of the possible extracted compounds listed above are the amines (NH₂), carbonyl group (COOH), hydroxyl (OH), sulfhydryl (SH), aldehyde (CHO), or azide (N₃) groups or a combination thereof. For instance, when isolating a peptide, amino acid groups from the peptide structure can be targeted. The intermediate linker is selected from a class of linkers that have at least two reactive ends, wherein one end has affinity to the targeted amino acids of the peptide, and binds to the peptide covalently, and the second reactive end can bind to specific functional groups to produce the engineered peptides. Various conformations of functionalized bio-polymers can be obtained, such as bio-polymer chains, nanoparticles/microparticles, nano bio-polymers or their gels. Suitable linkers include but are not limited to the linkers shown in Table 3.

TABLE 3
Preferred Covalent Functionalization of
the Extracts using Linker Intermediates

| Linker Reactivity | ExtractsTargeted | |
|---|---|---|
| Class | Groups | Linker Reactive End Group(s) |
| Amine-reactive | —NH₂ | NHS ester |
| | | Imidoester |
| | | Pentafluorophenyl ester |
| | | Hydroxymethyl phosphine |
| Carboxy-to-amine | —COOH | Carbodiimide (e.g., EDC) |
| reactive | | |
| Sulfhydryl-reactive | —SH | Maleimide |
| | | Haloacetyl (Bromo-o or Iodo-) |
| | | Pyridyldisulfide |
| | | Thiosurfonate |
| | | Vinylsulfonate |
| Aldehyde-reactive | —CHO | Hydrazide |
| (carbonyls) | | Alkoxyamine |
| Photo-reactive, | random | Diazirine |
| random insertion | | Aryl azide |
| Hydroxyl-reactive | —OH | Isocyanate |
| | | Dvinyl sulfone (DVS) |
| | | 1,4-butanediol diglycidyl |
| | | ether(BDDE) |
| Azide-reactive | —N₃ | Phosphine |

Keratin is recognized for its reactive cysteine amino acids. Covalent bonding of cysteine with amine-modified functional molecules can be performed in the presence of a linker, wherein the linker can be selected from: maleimide, haloacetyl (bromo or iodo-), pyridyldisulfite, thiosulfate, and vinylsulfone linkers. The resulting bioactive compound-keratin chains, nanoparticles or gels, can be used as a bioactive delivery system to enhance plant growth and development, to provide support during harsh environmental conditions, to treat various pathogens, or a combination thereof.

The engineered peptide can be synthesized using non-covalent bonds to the initial peptide structure, wherein a negatively charged functional group preferentially interacts with a cationic group, and a positively charged functional group preferentially interacts with an anionic group. The surface charge, size and conformation of the complex can be modulated by varying the concentration of the functional group. An initial peptide, with a specific pKa and surface charge, is added to a solvent or aqueous solution at a pH between 4.5-8, wherein the pH is selected to be above the pKa of the initial peptide. A different surface charge functional group is added to the peptide solution, and the mixture is reacted at room temperature between 10-30 min. The mass ratio peptide:functional group is chosen and modulated based on the desired final peptide or nanopeptide, z-potential, size, and stability. The ratio of functional groups to the initial peptide can be between 1:1 to 1:50,000 (w/w). The functional groups can be both hydrophilic and hydrophobic and include but are not limited to linkers, another peptide, polymers or copolymers (natural or synthetic), a nanoparticle, a bioactive molecule, or a combination thereof. Bio-polymers or nano bio-polymers having increased stability, can be produced by attaching a PEG tail to the peptide. Bio-polymers or nano bio-polymers having anti-fouling and anchoring properties to the plant leaves, can be produced by attaching an anti-microbial peptide functional group or a polymer, resin or another component that acts as anchoring agent to form superior adhesiveness to the plant leaves

Bio-polymer or nano bio-polymer delivery system compositions that carry a bioactive molecule can be customized to accommodate various amounts, or combinations of bioactive ingredients, by modulating the initial peptide type, size, molecular weight, and the ratio of peptide: bioactive molecule: functional group.

The functional groups of the invention can be engineered before the attachment to the initial peptide, and one or more distinct functional groups can be attached through a covalent or non-covalent bond.

The engineered bio-polymers can also carry biological molecules attached to its surface. In the case of nano bio-polymers, the biological molecules or the polymer/copolymer, or both can be attached on the surface of the nano bio-polymer, can be encapsulated inside the nano bio-polymer, or both. The biological molecule can be entrapped in the bio-polymer or nano-biopolymer by inserting them into a solution containing the biological molecule(s), wherein its absorption is controlled by pH, followed by the swelling of the engineered nano bio-polymer or gels. The biological molecules can be attached using linkers (covalent bonding) or by non-covalent bonding. Polymers or other biological molecules can be attached to the initial extracted bio-polymer, either before the non-covalent interaction with the functional group, or after the non-covalent interaction.

The initial bio-polymers and engineered bio-polymers can be used as a platform delivery system for the delivery of at least one bioactive agent. Bio-polymers extracted from various sources can be produced in various sizes, with different surface charges, different conformations, and in different compositions tailored for specific applications related to the function of the bioactive agent.

The loading capacity (LC) and encapsulation efficiency (EE) is calculated based on the formulas below. Using the EE varies between 30-62% depending on the method chosen, initial peptide and the final composition.

$$\begin{matrix}LC = \frac{Weight of AMO in KNPs}{Weight of KNPs} \times 100 \% & ( 1 ) \\ EE = \frac{Loading capacity of KNPs}{Loading capacity of KNPs in theory} \times 100 \% & ( 2 )\end{matrix}$$

A non-covalent crosslinking reaction can be performed by attaching a functional group from a class of metal complexes to the surface of the bio-polymer. For example, a phosphate precursor (calcium phosphate, sodium tripolyphosphate (TPP), etc.) can be attached using non-covalent interaction. Using TPP, the extracts from seafood chitosan can be synthesized to encapsulate various bioactive agents to treat a plant or to enhance its growth. The mass ratio chitosan: TPP can be 10:0.5, preferably 5:1, and the chitosan to encapsulated bioactive agent ratio can be 5:0.3-1. The resulting average nanoparticle size is between 20-400 nm, increasing with the bioactive agent concentration.

Nano bio-polymers are size tunable; can be multi-functionalized with a variety of functional molecules, such as linkers, peptides, DNA, proteins, polymers, bioactive molecules, or a combination thereof; can be synthesized using green chemistry, in a cost effective way, in high quantities; in a single step, or in a multistep process (depending on the composition). They can be surface coated with anchoring peptides to enhance their attachment to a plant leaf; they can be coated with a specific polymer (e.g. poly styrene-4-sodium sulfonate) or molecules or peptides to rapidly traverse the plasma membrane to target cellular organelles. The nano bio-polymers can include one or more types of metal nanoparticle, or functionalized metal nanoparticle (including a metal nanoparticle and a functional group), they can be encapsulated in the nano bio-polymers or attached to its surface or both. Further, they can be used in connection with a florescent molecule, or an imaging agent. The nano bio-polymers of the invention can be used for plant growth and protection and/or for intracellular labeling in a plant. Further, to increase the efficiency of the intracellular labeling, targeting molecules, such as antibodies and conjugated peptides can be attached. Exemplary targeting molecules include but are not limited to ACLSV, Ramularia collo-cygni, AMV, GLRaV-1, RBDV, A. niger, GLRaV-3, RSPaV, ApMV, GVA, SLRV, ArMV, GVB, SMYEAV, ASGV, OLRSV, TMV, CTV, OLV-1, ToRSV, GFKV, PDV, TRSV, GFLV, PNRSV, TYLCV.

### Agricultural Formulations

The bio-polymers and nano-biopolymers of the invention are used as novel effective smart materials or ingredients for sustainable intensification in agriculture to improve crop production and to manage plant disease. Bio-polymers, engineered bio-polymers, and micro or nano bio-polymers can be used to stimulate plant growth and development, stimulate root growth, increase adsorption of nutrients, increase crop yield, improve quality and taste of crops, increase amino acid or other nutrient content, and for protection against various pathogens. Bio-polymers and nano-biopolymers can be included in agricultural formulations and nanoformulations, such as fertilizers, pesticides, and herbicides. The compositions containing bio-polymers and/or nano-biopolymers can be used in certified organic farming that uses zero synthetic chemicals since the extracts are organic and have no adverse effect on the environment or humans.

The invention provides a process for making and customizing an agricultural active ingredient and an agricultural formulation. The agricultural formulation can be made in a one pot process as described in the following steps.

### Step 1. Initial Bio-Polymer Extraction

Solid waste is combined with a natural eutectic solvent (DES/NADES) to extract the bioactive polymer. The bio-polymer can be a peptide, polysaccharide or lipid.

### Step 2. Separating the Bio-Polymer

The extracted bio-polymer can be used in an agricultural formulation without further processing or the extracted bio-polymer can be separated from the reaction liquid by precipitation with organic solvents, followed by filtration, to isolate a pure bio-polymer. The extracted/isolated bio-polymer can be combined with various other bioactive components and used as an organic or conventional fertilizer.
Step 3. Engineering the Bio-polymer. Optionally, the bio-polymer extracts of Step 1 can be further engineered, either in the same liquid solution; or the solution can be separated and the bio-polymer can be re-dispersed in an aqueous solution, or other solution (different NADES, or buffered aqueous solution). The bio-polymer extracts can be combined in a dry form with a functionality group to produce an engineered enhanced bio-polymer, a nanowire, a nanoparticle, or a delivery system. The engineered bio-polymer delivery system can be utilized as an intelligent fertilizer, herbicide, pesticide, or insecticide, or a combination thereof.
Step 3A. Produce Nano bio-polymers using sonication. After sonication, the nano bio-polymers can be used in an agricultural formulation or nanodelivery systems can be produced by adding a bioactive agent, followed by additional sonication to encapsulate the bioactive agent inside the nano bio-polymers. The nano bio-polymer delivery system can be utilized as an intelligent fertilizer, herbicide, pesticide, or insecticide, or a combination thereof.

### Step 3A′. Multi-Responsive/Intelligent Nano Bio-Polymer Delivery System.

The nano bio-polymers produced by sonication can be used in an agricultural formulation or they can be further engineered by attaching specific anchoring, targeting, pH responsive and/or thermosensitive molecules to its surface. The resulting multi-responsive nano bio-polymers can be used in a multi responsive intelligent fertilizer, herbicide, pesticide, and/or insecticide.
Step 3B. Bio-Polymers with Ball Milling.
The extracted bio-polymer of Step 2 can be subjected to ball milling to produce nano bio-polymers. The resulting nano bio-polymers can be used in agricultural formulations or used to produce nanodelivery systems.
Step 3B′. Nanodelivery Systems or Multi-Responsive/Intelligent nano bio-polymers Delivery System. The nano bio-polymers of Step 3B can be used to encapsulate a bioactive by simply adding the desired bioactive compound to the ball milling solution. The resulting nanodelivery system can be used in agricultural formulations.

### Step 3B″ Multi-Responsive/Intelligent Nano Bio-Polymer Delivery System.

The nano bio-polymers produced by ball milling can be used in an agricultural formulation or they can be further engineered by attaching specific anchoring, targeting, pH responsive and/or thermosensitive molecules to its surface. The resulting multi-responsive nano bio-polymers can be used in a multi responsive intelligent fertilizer, herbicide, pesticide, and/or insecticide.

The bio-polymers can be used directly in agricultural formulations or combined with other components. In addition to bio-polymers and nano-biopolymers the agricultural formulations can include a) other nanocomposites (e.g. zinc oxide, titanium oxide, copper oxide and/or any metal nanoparticle form of the disclosed microelements), b) other bioactive compounds including but not limited to pesticides, herbicides, bactericides, fungicides, insecticides, acaricides, miticides, nemanticides, molluscicides, growth regulators, and sources of N, P and/or K, c) compounds which provide sources of carbohydrates (sugars, molasses, etc.), d) macronutrients including but not limited to magnesium, calcium, sulfur, and/or their precursors in the form of metal salt or a nanoparticle, e) metals including but not limited to Bo, Fe, Mn, Zn, Co, Cu, Mo, and/or Mg or precursors thereof, and/or f) other beneficial compounds including but not limited to carbon nanoparticles, carbon nanotubes, graphene, aluminum, silicon, or precursors thereof, in their bulk form or in nanoform. The agricultural formulations can be used to enhance plant growth and development and to protect the plant against various pathogens or a combination thereof.

Agricultural fertilizer formulations can contain a mixture of bio-polymers and nano-biopolymers and essential plant element precursors. Various concentrations and ratios of bio-polymers and nano-biopolymers can be combined with one or more of the 17 essential elements as listed in Table 5. Preferably, fertilizer agricultural formulations include nitrogen (N), phosphorus (P) and potassium (K). The nutrient input attributed to applying the bio-polymers and nano-biopolymers fertilizer on crops results in an increase in crop yield between 30% to 80%.

TABLE 5
Essential plant nutrient elements and
their primary form utilized by plants.

| | | |
|---|---|---|
| Essential plant element | Symbol | Primary form |

Non-Mineral Elements

| | | |
|---|---|---|
| Carbon | C | CO₂ (g) |
| Hydrogen | H | H₂O (I), H⁺ |
| Oxygen | O | H₂O (I), O₂(g) |

Mineral Elements

| | | | |
|---|---|---|---|
| Primary | Nitrogen | N | NH₄⁺, NO₃⁻ |
| Macronutrients | Phosphorus | P | HPO₄²⁻, H₂PO₄⁻ |
| | Potassium | K | K⁺ |
| Secondary | Calcium | Ca | Ca²⁺ |
| Macronutrients | Magnesium | Mg | Mg²⁺ |
| | Sulfur | S | SO₄²⁻ |
| Micronutrients | Iron | Fe | Fe³⁺, Fe²⁺ |
| | Manganese | Mn | Mn²⁺ |
| | Zinc | Zn | Zn²⁺ |
| | Copper | Cu | Cu²⁺ |
| | Boron | B | B(OH)₃ |
| | Molybdenum | Mo | MoO₄²⁻ |
| | Chlorine | Cl | Cl⁻ |
| | Nickel | Ni | Ni²⁺ |

Bio-polymers and nano-biopolymers can be added to conventional N,P,K fertilizer formulations at a concentration between 5-25% hydrolyzed peptide, preferably 5%-10%. Carbohydrate compounds can be included at a concentration of about 1%. The carbohydrate compounds are preferably derived from industry by-products, and include but are not limited to molasses, humic acid, polyglucuronic acid, and pectin. The polyglucuronic acid and pectin can be hydrolyzed in basic media for 30 min-2 hours at 40-60° C. before adding to the formulation. The fertilizer formulation can also include 0.1%-0.5% microelements such as Bo, Fe, Mn, Zn, Co, Cu, Mo, Mg, and preservatives. Preferably, the sources of nitrogen, phosphorus and potassium are organic such as yeast extracts. The pH of the fertilizer is preferably 2.5-4 to reduce microbial attacks.

A N, K, P fertilizer can be formulated to include 1) an initial bio-polymer that is engineered to nano bio-polymers and encapsulated with a N source to form a nanocomposite, 2) an initial bio-polymer that is engineered to nano bio-polymers and encapsulated with a K source to form a nanocomposite, 3) an initial bio-polymer that is engineered to nano bio-polymers and encapsulated with a P source to form a nanocomposite. The nanocomposites can be combined with one or more nanoparticles, including but not limited to zinc oxide, copper oxide, iron oxide, titanium oxide, calcium oxide, nickel, aluminum, silicon, and carbon. The nanoparticles can be in the solution, attached to the nano bio-polymers, or a combination thereof. The concentration and ratio of encapsulated N, K, and P is customized based on the plant's needs. The N, K, P fertilizer can include 5-75% phosphorus, preferably 55%; 5-55% nitrogen, preferably 25%, and 5-75% potassium, preferably 20%. The nanocomposite sizes can be between 10-1000 nm, but are preferably between 100-200 nm. Additionally, the nanoparticles can be of different sizes and shapes such as spherical, cubic, rod, rhombic, wires, chains, etc.

An N organic fertilizer can be formulated containing 1) 5-25% selected hydrolyzed bio-polymers; 2) 2-10% enzyme extracts, and 3) 1-3% carbohydrates. The bio-polymerscan further contain an anchoring peptide on its surface to increase attachment of the fertilizer to the hydrophobic leaves of the plant. The fertilizer can be applied in a liquid form at an adjusted pH of 2-4.

An N, K, P organic fertilizer can be formulated containing 1) cross-linked initial extracted bio-polymer gels, wherein the bio-polymer is in an amount between 5-35%, preferably 5%-10%, 2) one or more bioactive compounds absorbed inside the bio-polymer gel, wherein the bioactive compounds include N, K, and/or P, 3) optionally, yeast extract in an amount between 2-13%; and 4) digested carbohydrates in an amount of 1%. Optionally, the bio-polymer can comprise anchoring peptides for enhanced attachment and internalization to the plant. The fertilizer can be in a liquid, powder, or gel form and may be formulated for quick or controlled release.

An organic N,K,P nanofertilizer can be formulated containing an engineered bio-polymer in the form of nano bio-polymers in an amount of about 5-35% bio-polymer, preferably 15-30%, and at least one bioactive compound encapsulated with the nano bio-polymer in a nanoparticle. The bioactive compound includes an organic certified source of N, P and/or K. The engineered bio-polymer can optionally include at least one anchoring and/or targeting group attached to the surface of the nanoparticle. The N, K, P nanofertilizer can be applied after appropriate dilution of 1:900-1:400 depending on the plant to be treated. The nanoparticles or can be further cross-linked to form a nanofertilizer gel. The nanofertilizer can optionally include one or more digested carbohydrate compounds in a concentration of about 1%. Suitable digested carbohydrate compounds include but are not limited to molasses, humic acid, polyglucuronic acid and/or pectin. The carbohydrate compounds are digested prior to inclusion in the nanofertilizer using digesting enzymes selected from: pectinase, mannanase, cellulase, xylanase and/or amylase. Optionally, a yeast extract can be included at a concentration of about 10-13% and microelements such as Bo, Fe, Mn, Zn, Co, Cu, Mo, Mg and/or a precursors thereof can be included at a concentration of 0.1%-0.5%, preferably 0.2%. The microelements can be added in their nano form, as a metal salt, or a combination thereof. The micro/nano elements can be cross-linked to the surface of the nanoparticles, cross-linked to the gel, and/or can be added to the nanoparticle solution as a mixture. The size of the nanoparticles can be from 5-1000 nm, and their shape can be spherical, cubic, rods, dimers, trimers, quatramers, rhombic, or a combination thereof.

Anchoring peptides can be used to improve the delivery of the bio-polymers and nano bio-polymers into the plant after application, preferably after foliar application. The anchor peptide can have between 10 to 100 amino acids. The mass fraction of the anchor peptide is between 0.2-2 mol %. As an example, the anchoring peptide can be selected from a class of anti-microbial peptides including plantaricin A. Plantaricin A (PlnA; 26 aa) in an amount of 0.45 mol % can be conjugated onto nanoparticles derived from keratin hair extracts. Plantaricin A increases attachment of the nano particles to the surface of the plant leaves as compared to non-conjugated nano particles. Plantaricin A—keratin nano particles remain attached to the leaves, even when the surface of the leaf is washed.

Bio-polymers and nano-biopolymers on plant leaves or within the endosomes have been found to show slight agglomeration. Keratin nano bio-polymer delivery systems, produced by ball milling bio-polymers and nano-biopolymers, are very stable between pH 7-9, but tend to agglomerate at lower pH values ˜ 5.3. The keratin nano bio-polymers can be strategically prepared at this pH value. When applied to a plant leaf, where the pH is 5.6-5.8, they internalize into the plant without releasing the encapsulated bioactive agent on the surface of the leaf. Disintegration and release of the bioactive agent is achieved enzymatically due to the glutathione inside the cells. In certain embodiments, in order to prevent agglomeration of the bio-polymers and nano-biopolymers, strategic polymeric functional groups such as PEGOH-CPP can be attached to the initial peptide structure. When the PEG moiety has a silyl group or an anti-microbial peptide attached to one end, the resulting delivery system can anchor to the waxy surface of the plant leaves.

Handling and management of industry by-products is costly because they require significant space for storage, and management. There are also significant sustainability concerns. However, using the present methods, these by-products can be utilized as raw materials to produce renewable bio-NADES. The resulting bio-NADES (slurry or pure liquids) can then be used in the same pot process to extract other agricultural ingredients from various sources of waste. The one pot method is a platform method, it is multifunctional, and can use NADES, bio-NADES, DES or a combination thereof to extract agricultural ingredients from various types of waste. Various agricultural products can be produced, and they can be presented in liquid, powder and/or gel form.

Wool or feather by-products were chosen as a keratin peptide source for the examples because of their low processed quantities as compared to other peptide by-products. Keratin by-products are one of the most abundant bio-wastes. Only a small percentage of keratin by-products are processed to biomass and re-utilized in industries such as cosmetics, pharmaceuticals, animal feed, and agricultural formulations. The low keratin quantities processed vs the waste quantities produced, make keratin by-products a potential threat to the environment. Poultry feathers alone generate about 8.5 billion tons of keratin bio-waste annually, however, only about 40 million tons of keratin bio-waste are processed every year. The remaining keratin bio-waste is dumped, buried, or incinerated producing environmental hazards, pollution, negative effects on human health, and increased greenhouse gas concentrations.

The following examples are provided to enable those of ordinary skill in the art to make and use the methods and formulations of the invention. These examples are not intended to limit the scope of what the inventor regards as the invention. Additional advantages and modifications will be readily apparent to those skilled in the art.

### EXAMPLES

### Example 1 Biomass Extraction Using Natural Eutectic Solvents (NADES)

The hydrogen bond donor (HBD) is chosen for DES/NADES synthesis based on their acidity and the viscosity of the final DES. The strength of the hydrogen bond between HBA:HBD influences the potency of the DES/NADES, and increases with their acidity. Oxalic acid is preferred as a HBD, based on its strong acidity. Natural acids such as lactic acid, acetic acid, formic acid, and citric acid can also be used. Choline chloride is dried under vacuum a.t 80ºC for 48 hours before use. The oxalic acid (HBD) was mixed the choline chloride at a preferred molar ratio HBD: ChCl of 0.5:1 in an iodine flask, avoiding any contact with air moisture, and heating at 80ºC, until a homogenous and transparent liquid is obtained.

The solid waste, 2 g of ground poultry feathers, is dissolved in 200 ml ChCl-OA (DES/NADES) at a weight ratio 1:1, and mixed for 6 hours at a temperatures of 60° C. The extract is filtered, and the supernatant precipitated with ethanol. The precipitate is then filtered and frozen for 3 h. The resulting keratin peptide extracts have a molecular weight of about 12 KDa, about 96% purity and a 98% reaction yield.

The DES and NADES can be reused by evaporation of the ethanol in a high vacuum pump at 45° C.

### Example 2 One Pot Process for Biomass Extraction, Production of Nano Bio-Polymers and their Encapsulation with Bioactive Compounds

The peptide can be extracted following the procedure of Example 1 and then re-dispersed in an aqueous solution with a pH between 6-8 at a concentration between 0.5-5%. The resulting mixture is ultrasonicated at 800 W for 10-30 min to produce the peptide-based nanoparticles. A particle size between 1-1000 nm can be obtained by controlling the sonication time, the concentration of the extracted peptide and the pH. To obtain nano bio-polymers between 100 nm to 150 nm, the pH is 7, the sonication time t=10 min, and the extract concentration is 1%.

In the same reaction, the nano bio-polymers can be encapsulated with a bioactive agent by introducing the bioactive agent into the mixture at a nano biopolymers: bioactive agent mass ratio of 50:1 (w/w), and the mixture is subjected to further sonication for another 10-30 min.

The peptide extracts obtained according to the procedure of Example 1, are subjected to sonication without separating the ChCl-OA liquid to produce the nano bio-polymers in a one pot synthesis. Ultrasonication was applied at pH=7 for 10 min to produce keratin nanoparticles of 100 nm. The keratin nanoparticles were encapsulated with organic potassium and phosphorus by sonicating the mixture for another 10 min after adding the potassium and phosphorus. The encapsulated nanokeratin can be used in an agricultural formulation without further processing or it can be purified by centrifugation. The resulting encapsulated nanokeratin having about 18.96 μg/mg to about 22.75 μg/mg potassium per milligram of particle.

### Example 3 Encapsulation of Nano Bio-Polymers Obtained by Ultrasonication Using pH Controlled Swelling

The nano bio-polymers of example. 2 (after extraction and sonication) are isolated either by precipitation and filtration or by centrifugation, then added to a solution that contains the bioactive agent, under a specific pH. The pH is chosen so that the nano bio-polymers swell, and diffusion of the bioactive agent into the nano bio-polymers occurs, followed by a pH adjustment to shrink the encapsulated nano bio-polymers. When using the encapsulation by taking advantage of its swelling capability, the loading is performed at very acidic pH-3 or very basic pH-9 where the keratin nanoparticles are highly swollen. The nanoparticles are placed in the swelling solution containing the bioactive agent and continuously stirred for 2 hours. After, the loading solution pH is increased to about pH 6, the nano bio-polymers collapse and entrap the loaded bioactive agent within the polymer network. Using this procedure, the amount of potassium encapsulated is between 25.06 and 39.15 μg/mg particles. Note that co-encapsulation can be also performed using both encapsulation methods.

### Example 4 Synthesis of Nano Bio-Polymers Using a Crosslinking Method

100 mg of powder keratin extract made according to Example 1, are diluted with 2 ml of deionized water and then mixed with 8 ml of absolute alcohol or a mixture of AOT/1-HP/Isooctane (25%/62.5%/12.5%). To this, 1 ul of 8% glutaraldehyde, divinyl sulfone (DVS) or 1,4-butanediol diglycidyl ether (BDDE) is added. The mixture is stirred using a magnetic stirrer at 40 rpm for 24 h. The stirred content was then centrifuged by cooling centrifuge (Remi) at 10,000 rpm for 20 min. The supernatant is discarded, and the pellet is collected. A lyophilization process is performed to obtain the keratin nanoparticles. The resulting nanoparticles and nanogels can be encapsulated according to the procedure of Example 3.

### Example 5 Production of Chitosan Nano Bio-Polymers

A chitosan extract with a concentration of 35%, is diluted to 0.1% and the pH is adjusted to 5.5 while simultaneously mixing with a TPP solution at a concentration between 0.02-0.05% at pH-7.5 and a bioactive molecule at a concentration between 0.03 to 0.08%, using a programmable pump. The volumetric flow rates from 20-500 mL min_1

### Example 6 Hydrolysis of Waste By-products Using NaOH in the Presence of Microelements

100 grams of solid waste protein (bath ratio 1:1) are mixed with 100 ml of alkaline solution at a concentration of 5-20%. The solid waste protein is hydrolyzed by shaking or stirring the solution in the presence of 0.1 to 1 wt % concentration of microelements at to 60° ° C. between 30 min to 4 hours. The solution is neutralized to pH=7.5 and mixed at room temperature for another 20 min. Peptide chelates of Fe 3+ can be prepared by using the ammonium sulfate solution of iron. Peptide chelates of Zn2+ can be prepared using Zinc Chloride as a source of Zn2+. Peptide chelates of Mg2+ can be formed by addition of manganese sulfate. Peptide chelates of Cu 2+ can be formed by adding copper sulfate. The pH of the solution is adjusted to pH=6.5 to 8, preferably at 7.5 using sulfuric acid. The solution containing the peptide chelate compounds can be used directly as a fertilizer or it can be used as a fertilizer raw material subject to further processing.

### Example 7 Surface Functionalization of Bio-polymers and Nano Bio-polymers

The extracted bio-polymers and nano bio-polymers according to the Examples 1-6 can be further functionalized by using covalent functionalization or a covalent reaction using a linker.

Covalent functionalization of an extract with a desired molecule can be achieved using intermediate linkers, wherein the targeted groups of the extracts are either the amines (NH₂), carbonyl groups (COOH), hydroxyl (OH), sulfhydryl (SH), or aldehyde (CHO), or azide (N₃) or a combination thereof. Keratin possesses a number of pendant functional groups such as —NH₂, —COOH, —SH and —OH.

The amine functional groups of keratin bio-polymers or nano bio-polymers and the carboxyl functional groups of a desired compound (e.g. a bioactive agent, a polymer-acrylate or methacrylate, or 2-carboxyethyl acrylate) can be cross-linked. The components in a molar ratio 1:4:4 of an aqueous extract of keratin (12 KDa), a COOH-terminated functionality (ex: polymer or co-polymer (methacrylic acid)), and 1-ethyl-3-(3-dimethylaminopropyl)carbodiimide hydrochloride (EDC) are mixed at pH-4.5 and reacted for 1 h. The resulting cross-linked keratin is precipitated using ethanol and then freeze-dried to obtain the powder keratin-methacrylic acid. Any molecule having a COOH group can be attached to keratin or another peptide having a free NH2 group, using this method. Crosslinking between the amine functional groups of keratin and a carboxyl functional can be carried out using thiol click chemistry utilizing succinimidyl 4-(N-maleimidomethyl)cyclohexane-1-carboxylate (SMCC) as the cross-linker. An anchor-peptide can be bound at one end (or subsequently added) resulting in a keratin-anchoring peptide functionality.

An eGFP-anchor-peptide fusion protein, (0.45 mol % Plantaricin A (PlnA; 26 aa)) can be conjugated onto the keratin bio-polymers and nano bio-polymers of Example 1-6, and the resulting Plantarcin-bio-polymers and Plantarcin-nano bio-polymers can be applied to a plant by foliar application. Plantarcin A increased the attachment of the bio-polymers and nano bio-polymers to the surface of a plant leaf and remained attached to the leaves even when the leaf surface was washed. The keratin functionalization with 0.1 to 0.9 wt. % eGFP-anchor-peptide fusion protein is produced as follows: 20 g keratin extracts of Example 1-6, were dispersed in a aqueous solution of pH 7.5, and mixed with SMCC at a concentration of 1 mg/ml-10 mg/ml, preferably 1.5 mg mL-1, 20× equivalent with respect to the peptide and stirring for 15 min. Subsequently, the eGFP-anchor-peptide fusion protein (0.1 mg, MW: 32314.7 g mol-1) was added and the mixture was stirred for 2 h at room temperature, followed by precipitation using ethanol, and freeze dried overnight.

The keratin of Examples 1-6 are functionalized with biomolecules containing-NH2, and the reaction is carried out between molecule-NH2 and keratin-COOH. The reaction requires the activation of the carboxylic groups of COOH, using 1-Ethyl-3-(3-dimethylaminopropyl)carbodiimide (EDC) in the presence of N-hydroxysuccinimide (NHS). Keratin-COOH and the NHS are dissolved in distilled water and the pH is adjusted to 5.4 by an addition of 2 M NaOH solution. After 15 minutes of constant stirring, the EDC solid was added slowly to a molar ratio EDC/NHS/COOH of 10:4:1. The pH was kept constant at 5.4. Next, the peptides were precipitated using ethanol. The resulting compounds can be freeze dried, oven dry or dispersed in an aqueous solution at a preferred concentration. Using the EDC/NHS coupling chemistry, a variety of peptides can be cross-linked.

The engineered keratin of Examples 1-6 are formed by dissolving the keratin 1 mg/mL solution in an aqueous solution with a pH between 4.5-7.5. Depending on the amino acid composition of the keratin polypeptide, the solubility pH will be different. Then, SMCC is added at a concentration between 1 mg/ml-10 mg/ml and the reaction is mixed for at least 3 hours. Optionally, the SMCC linker can contain a bioactive compound either an agricultural active compound, a polymer or any desired compound having an amino group. The amino-terminated-compound can be added to the SMCC linker at a molar ratio 1:10 in 100 mM KH₂PO₄ buffer at pH 7.2 and mixed for at least 1 h. The resulting conjugate is then purified. The resulting conjugate can be a keratin chain or nanoparticles depending on the amino terminated compound.

Covalent reactions using a linker to hydroxyl (OH) functionalities; a linker to aldehyde (CHO) functionalities or using a linker to azide (N₃) functionalities can be performed on the extracts of Examples 1-6. A combination of surface functionalization can be performed as well.

### Example 8 One Pot Green Process for Producing an Organic Keratin Smart Delivery NanoFertilizer, NanoPesticide, NanoHerbicide, NanoFungicide or Nanoinsecticide, or a Multimodal NanoFormulation Using Renewable Bio-NADES

The process comprises the following steps: 1) making the bio-NADES; 2) extracting the keratin from feathers; 3) engineering the keratin using ball milling to nanoscale to produce nanofertilizers; and 4) adding bioactive compounds such as growth regulators, pesticides, herbicides, fungicides, or insecticides to step 3, to encapsulate them inside a keratin nanoparticle. A combination of two active ingredients can be added to step 3, and co-encapsulated. The resulting formulation will act as a dual modal formulation. Up to ten active ingredients can be added at step 3, to produce a multifunctional formulation.

Step 1. Making the bio-NADES: 30 kg of sugar cane bagasse (or molasses) can be ground and pretreated in a 100 L reactor in acidic media using ballmilling at 500 rpm for 2 h at 60ºC, pH=4. Next, half of the pretreated bagasse slurry, 50 L is placed in a fermentation reactor and incubated with 2% urea, 3% methanol and A. niger spores, 107 spores/g of dry material for 4 days. The fermentation reactor lid is punctured to facilitate and optimize oxygen penetration, at 75% moisture, as well to provide proper ventilation of the heat produced during the fermentation process. The remaining pretreated bagasse, 50 L is mixed with the fermenting half and the reaction is thoroughly mixed with ballmilling for an additional 30 min at 50ºC. The resulted is the bioNADES.

Step 2. In the same pot process, 1 to 10 kg of ground feathers is added to the ball milling reaction resulting in the extraction of keratin and other components from the feathers. The reaction is carried out at 65° C. for 2 hours with a resulting extraction efficiency of 85%. The resulting nanoslurry (100-130 nm) can be used as a nanofertilizer. In order to decrease the nanoparticle sizes, ballmilling can be carried out for an additional 30 minutes. Sonication can be performed at this step for 20 min to produce nanoparticles of less than 100 nm.

Step 3. Bioactive compounds having plant growth and protection functions, at a ratio of 0.1-1:10 active compound:keratin can be added after step 2 to produce a NanoFertilizer, or NanoPesticide, NanoHerbicide, NanoFungicide and/or a Nanoinsecticide.

### Example 9 Fertilizer Formulations

The keratin bio-polymers or nano bio-polymers can be added to conventional N, P, K fertilizer formulations. The formulations include (1) keratin or nanokeratin in a concentration between 5-25%, preferably 5%-10%; (2) 1% Molasses; (3) 0.1%-0.5% microelements such as Bo: 0.2-0.3 g/l; Fe: 0.3-0.5 g/l; Mn: 0.4-0.7 g/l; Zinc: 0.5-0.7 g/l; Co: 0.05-0.1 g/l; Cu: 0.2-0.3 g/l; Mo: 0.7-0.9 g/l; and Mg: 1.2-1.5 g/l; (4) P₂O₅ between 90-100 g/l; (5) urea between 150-170 g/l; (6) K₂O between 80-90 g/l; (7) 5% yeast extracts and preservatives. The pH of the fertilizer is kept to 4 to avoid microbial attacks.

A nanocomposite fertilizer of N, K, P, can be prepared which includes 1) Urea encapsulated nanoKeratin 2) P₂O₅ encapsulated nanokeratin, and 3) K₂O encapsulated nanokeratin. Optionally, the nanoKeratin composites can be combined with other nanoparticles, such as zinc oxide, copper oxide, iron oxide, titanium oxide, calcium oxide/hydroxide, hydroxyapatite nanoparticles, nickel, aluminum, silicon, carbon, precursor nanoparticles, or a combination thereof; wherein the other nanoparticles are attached to the keratin, are in the solution, or a combination thereof. Wherein, their concentration and ratio are customized based on the plant needs. The concentration is between 500 ppm to 4000 ppm, but preferably at a concentration lower than 1000 ppm in the final volume. It is preferable that the nanomicroelements be applied at a concentration of 15-20 times lower than the recommended field dosage of their bulk counterparts. The fertilizer formulation includes from 5-75% phosphorus, preferably 55%; 5-55% nitrogen, preferably 25%, and 5-75% potassium, preferably 20%. The nanocomposite sizes can be between 10-1000 nm, preferably between 100-200 nm. Additionally, the nanoparticles can be of different sizes and shapes such as spherical, cubic, rod, rhombic, wires, chains, etc. The nanofertilizer can include a mass fraction of the anchor peptide Plantaricin in an amount between 0.2-2 mol %, but preferably 0.45 mol %.

An N organic fertilizer is disclosed, comprising 1) 5-25% keratin extracts or nanoKeratin; 2) 2-10% enzyme extracts, and 3) 1-3% Molasses and preservatives. The keratin component can further include Plantaricin A in an amount between 0.2-2 mol %, but preferably 0.45 mol % on its surface. The result is a Smart Foliar nano N organic fertilizer with controlled delivery in the form of a liquid with an adjusted pH of 4. The resulting fertilizer can be also converted to powder, by freeze drying or other well know methods.

A fertilizer for metal deficiency crops can include the above discussed components and in addition, metal precursors of Bo, Fe, Mn, Zn, Co, Cu, Mo, Mg, or a combination thereof. The loading of the metal ions is performed by loading of the keratin bio-polymers and/or nanokeratin bio-polymers with metal ions, using a 4-fold excess (volume with respect to the keratin mass) of metal ion solution, under acidic pH˜2. Next, the mixture was diluted 4× and stirred at room temperature for 4 hours. Subsequently, the solution was centrifuged at 5000 rpm for 30 min to separate the nanokeratin or keratin from the unabsorbed ions. Nanoparticle precursors of Bo, Fe, Mn, Zn, Co, Cu, Mo, Mg, or a combination thereof can be absorbed. Their concentration and ratio are customized based on the plant needs, are between 500 ppm to 4000 ppm, preferably at a concentration lower than 1000 ppm in the final fertilizer volume.

Example 10 A N, K, P organic fertilizer gel can be produced in a one pot synthesis. The fertilizer includes: 1) Keratin bio-polymer gel and nanokeratin gel, at a concentration of 5-35%, but preferably 5%-10%, that was synthesized according to the above examples: using NADES for extraction, and then subsequently adding nitric oxide to crosslink the keratin and form gels. The resulting nanokeratin gels are 50 nm. Further, the bioactive compounds N, K, P are absorbed into the gel. The N, K, P composition and ratio is selected from a range between from 5-75% phosphorus, but preferably 55%; and 5-55% nitrogen, but preferably 25%, and 5-75% potassium, but preferably 20%. In a further embodiment, microelement and nanoelements are added using the absorption method described above. The one or more additional nutrient can be a part of the composition that comprises the nanoparticle or can be a part of the nanoparticle, or engineered peptide, or their gels.

### Example 11

A seed priming or seed coating composition can be synthesized using a dry chemical method. First, keratin was extracted using a Urea:K2SH system that was formed by adding 1.2 g of Urea, 74 mg of K2SH and 10 ml of water and reacting it at 80° C. until a white liquid was formed. Then, 130 g of feathers were added to the liquid and the extraction was carried at 80° C. for 1.5 hours. The resulting keratin size was of 10 KDa. Without any further purification, microelements in the form of oxides or chlorides of Ca, Mg, Mn, or Zn in a percentage of 2-4% of the total keratin were added to the composition. Anti-freeze components and Choline chloride-EG DES of 0.2-1% were added. Gentian violet was added as a colorant in the waxy composition. Finally, the seeds were immersed in the composition in a volume ratio of 0.2 ml/seed, having a peptide concentration of about 1-5%. FIG. 3R shows the seeds before and after coating. In order to improve the preservation, a second protection layer can be added on the top of the active composition. The second protection layer can be for example, gum Arabic, clay, polyacrylic, PEG, or starch or a combination thereof.

### Example 12

Dicamba herbicide can be encapsulated using the dry synthesis method. Dicamba and keratin were added in a ratio of 1:3 with the reaction initiated using 5% water, and the subsequent addition of a functional linker, 5% oleylamine, followed by precipitation of the resulting waxy product using different pHs and buffers (see FIGS. 3A-D). Precipitation using Tris buffer 10 mM, at pH 7 (FIG. 3A), produced 100 nm spheres vesicles; at pH 6.5 (FIG. 3B), produced spheres of about 100 nm; at pH 4.5 (FIG. 3C) produced nanoparticles around 200 nm; at pH 3.5 (FIG. 3D) produced polydispersed nanoparticles of different sizes; at pH 8 (FIG. 3E) produced nanoparticles of around 50 nm in size; at pH 10, the nanoparticles seemed to have an undefined shape. Other solvent precipitants at pH 7 were also tested and their impact on size and shape are shown in FIGS. 3G-J as follows: methanol (FIG. 3G), Tap Water (FIG. 3I); Acetone, (FIG. 3H); Acetone+Methanol 1:1 (FIG. 3J).

Different nanoparticle shapes and sizes were produced by using different fatty functional linkers such as calcium stearate, iron stearate, and others. The calcium stearate butterfly shape is shown in FIG. 3M, the rice shape is shown in FIG. 3N and the sphere shape is shown in FIG. 3N′. The dry chemistry functional linker can also be a polymer. Representative TEM images of PEI are shown in FIGS. 3O-P.

### Example 13

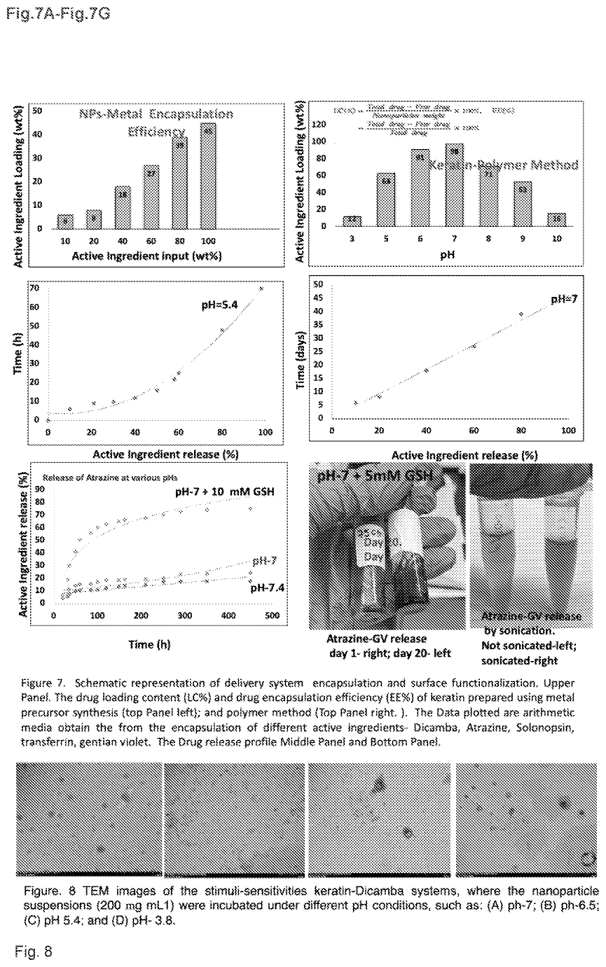

Drug loading capacity using both metal encapsulation and the dry polymer method was evaluated. The encapsulates were dialyzed for 1 day to remove unstable adsorbed drug. Subsequently, the free drug was measured by UV spectrometry. Further the dialyzed delivery systems were freeze dried to obtain a pure encapsulated delivery system. The drug loading content (LC %) and drug encapsulation efficiency (EE %) were calculated according to the equations presented in FIG. 7. The most effective drug loading was performed at pH 7, with about 98% encapsulation efficiency. 91% loading efficiency was obtained at pH 6.5.

### Example 14

A keratin-herbicide (dicamba) pH control delivery system was produced. The system reduces volatilization of dicamba, confers increased efficiencies, reduces the amount of herbicide application, reduces human exposure, and reduces toxicity. The delivery system was encapsulated using the dry method, keratin:dicamba ratio 1:10 weight ratio, initiated with 10% water and 5% oleylamine. The waxy clay was precipitated by the addition of tap water having 0.1% Tween 20, at pH 7. The resulting nanoparticle delivery systems were about 100 nm in size and were observed by the appearance of a white milky colored solution. The TEM images of the resulting nanoparticles are shown in FIG. 8A. The pH controlled release was demonstrated by TEM imaging and Z-potential. The nanoparticles were centrifuged and exposed to different water solutions for four hours. FIG. 8B shows the results at pH 6.5, the nanoparticles were observed to agglomerate and leach in small amounts. FIG. 8C shows the delivery system exposed to pH 5.4, dark patches can be seen along with half full vesicles surrounded by leached herbicide. FIG. 8D shows the results at pH 3.5, the nanoparticles swell to 1-2 micron from their initial 100 nm, and the herbicide leach is accentuated and appears as big grey patches all over the grid. In addition, a time release profile of dicamba at pH 5.4 and pH 7 was assessed and the results are presented in FIG. 7 middle panel. There was a complete release of Dicamba at pH 7 in 45 days; and a complete release of dicamba at pH 5.4 in about 70 hours.

### Example 15

The delivery systems according to the present invention can be used for an enzymatically controlled release. In plants, glutathione is found in the cytosol, endoplasmic reticulum, vacuoles, mitochondria, chloroplasts, peroxisomes, and the apoplast (Noctor and Foyer 1998). For example, in tobacco mesophyll cells, GSH is distributed as 76%, 17%, and 7% in chloroplasts, vacuoles, and the cytoplasm, respectively (Berthe-Corti et al. 1992). Some plant species (e.g. soybean, pea, peanut) do not primarily contain GSH; instead, they contain another thiol called homoglutathione (γ-glutamylcysteinyl-β-alanine), which functions in a similar way to GSH and shows the same redox reaction. Keratins are known to be GSH and HGSH responsive, by their exposure the nanoparticle system will deliver the encapsulated compounds in an enzymatically control manner.

The enzymatic release profile of the nanoparticles was evaluated using the dialysis method at 37° C. The nanoparticles were exposed to different concentrations of GSH, 10 mM and 50 mM, and at certain times, the dialyzed encapsulate was tested by UVspectrometer.

The herbicide atrazine combined with gentian violet was encapsulated using the dry method at a weight ratio of keratin:atrazine of 1:15 and a weight ratio of keratin:GV of 1:0.5. The water quantity was 5% in each and the functional molecule was fatty hexylamine. The control release properties were stimulated both enzymatically via glutathione (GSH) 10 mM, and by pH as shown in FIG. 7, lower left panel. A complete release at pH 7 when they were stimulated enzymatically with 10 mM GSH in 500 h vs only about 30% release at pH 7 with no enzymatic stimulation, and 15% release at pH 7.4. The delivery systems were shown to have a GSH concentration dependent release profile with a complete release of the drug in 300 h when exposed to 50 mM GSH. The nanoparticles were also shown to be responsive to sonication (See FIG. 7, bottom right panel). Showing the loss in in color intensity with the release of the GV.

### Example 16

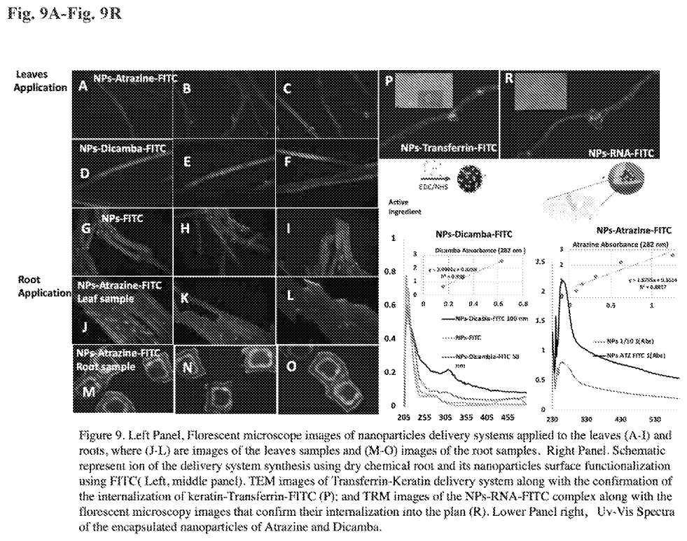

Using a delivery system, an active agricultural compound, such as a nutrient, herbicide, pesticide, peptide, RNA, DNA, a complex of enzymes with DNA or RNA, or a combination thereof can be complexed with the bio-polymer and delivered to a plant. A delivery system of keratin extracted from feather waste was encapsulated with an herbicide and applied to a pepper plant both by foliar and root application. FIGS. 9A-O show the pepper plants treated with the delivery systems. Pepper plants treated by foliar application with NPs-Atrazine-FITC are shown in FIGS. 9A-C; pepper plants treated with NPs-Dicamba-FITC are shown in FIGS. 9D-F; and pepper plants treated with NPs-FITC are shown in FIGS. 9G-I. Root application with NPs-Atrazine-FITC are shown in FIGS. 9J-L (cross section of leaf) and FIGS. 9M-O (cross section of root). In all cases the green florescence indicates the internalization and distribution of the nanoparticles inside the plant. The herbicides first tagged with a florescent molecule, floresceinisocyanate (FITC) using the known EDC/NHS coupling. DCC (1.62 mmol) was dissolved in DMF (25 mL) and added dropwise to a solution of Dicamba (3.24 mmol) and NHS (3.24 mmol) in DMF (25 mL) under nitrogen atmosphere with ice-cooling. The mixture was stirred at low temperature overnight to form a white precipitate followed by filtration. Appropriate amounts of the filtrate were added to a solution of 2 mg/ml of FITC at pH 8.0. The herbicide conjugated FITC and FITC alone were then encapsulated into the keratin bio-polymer to form a florescent delivery system.

### Example 17

The delivery systems keratin-transferrin-FITC, and NPs-RNA complex were synthesized in a weight ratio of 1:0.5, keratin: transferrin, and 5:1 keratin: RNA, followed by the addition of 5% water and 8% calcium-stearate modification. The waxy product was precipitated to nanoparticles by using 10 mM Tris pH 8. The transferrin and RNA was modified with FITC using EDC/NHS coupling. The delivery system was applied to the plant by foliar application. FIGS. 9P-R show the TEM images of the complex, keratin-transferin (FIG. 9P left corner), Keratin-RNA (FIG. 9R left corner), along with their internalization into the plant (FIG. 9P—transferrin complex internalization, FIG. 9R—Keratin-RNA internalization). The internalization of the complex is represented by the florescence.

1. A method for recycling bio-waste into an agricultural composition, comprising:
a) selecting a bio-waste based on the intended application of the agricultural formulation,
b) mixing the bio-waste with at least one natural eutectic solvent to extract at least one biopolymer selected from the group consisting of peptides, polysaccharides and lipids,
c) reducing the size of the extracted bioactive peptides to produce nanopeptides of different sizes and shapes,
d) adding at least one additional bioactive agent during step c) to produce a chain bio-polymer delivery system and/or an encapsulated nano bio-polymer delivery system, and
e) applying the chain bio-polymer delivery system and/or encapsulated nano bio-polymer delivery system to a plant as an agricultural composition.

2. The method according to claim 1, wherein said bioactive agent is selected from the group consisting of nanocomposites, pesticides, herbicides, bactericides, fungicides, insecticides, acaricides, miticides, nematicides, molluscicides, growth regulators, sources of nitrogen, phosphorus, potassium, carbohydrates, magnesium, calcium, sulfur, precursors in the form of a metal salt or a nanoparticle, and metals.

3. The method according to claim 1, further comprising attaching functional groups to the surface of the chain bio-polymer delivery system and/or encapsulated nano bio-polymer delivery system.

4. The method according to claim 3, wherein said functional groups are selected from the group consisting of anchoring peptides, targeting peptides, pH responsive molecules and thermosensitive molecules.

5. The method according to claim 4 wherein said functional group is a plantaricin A anchoring peptide.

6. The method according to claim 1, wherein the chain bio-polymer delivery system and/or the encapsulated nano bio-polymer delivery system has a diameter between 1-1000 nm.

7. The method according to claim 1, wherein the size and/or shape of said extracted bio-polymer is modified by adding a functional group via a dry or wet chemistry method or by using sonication or ball milling.

8. An agricultural composition comprising chain bio-polymers and/or nanoparticles, wherein said chain bio-polymers and nanoparticles have at least one functional group on their surface.

9. The agricultural composition according to claim 8, wherein said functional group is selected from the group consisting of anchoring peptides, targeting peptides, pH responsive molecules and thermosensitive molecules.

10. The agricultural composition according to claim 8, wherein said anchoring peptide attaches the chain bio-polymers and/or nanoparticles to the surface of a plant.

11. The agricultural composition according to claim 10, wherein said anchoring peptide is plantaricin A.

12. The agricultural composition according to claim 8, further comprising at least one additional component selected from the group consisting of nanocomposites, pesticides, herbicides, bactericides, fungicides, insecticides, acaricides, miticides, nemanticides, molluscicides, growth regulators, sources of nitrogen, phosphorus, potassium, carbohydrates, magnesium, calcium, sulfur, their precursors in the form of metal salt or a nanoparticle, and metals.

13. The agricultural composition according to claim 8, wherein a bioactive agent is encapsulated in said nanoparticles, attached to the surface of said nanoparticles or a chain bio-polymer, and/or attached between two chain bio-polymers.

14. The agricultural composition according to claim 13, wherein said encapsulated bioactive agent is selected from the group consisting of nanocomposites, pesticides, herbicides, bactericides, fungicides, insecticides, acaricides, miticides, nemanticides, molluscicides, growth regulators, sources of nitrogen, phosphorus, potassium, carbohydrates, magnesium, calcium, sulfur, their precursors in the form of a metal salt or a nanoparticle, and metals.

15. The agricultural composition according to claim 8, wherein said nanoparticles have a diameter between 1-1000 nm.

16. The agricultural composition according to claim 8, wherein said bio-polymers are a protein, polysaccharide, or a natural polymer.

17. The agricultural composition according to claim 8, wherein said bio-polymers are derived from keratin, collagen, chitin, chitosan or lignin.

18. The agricultural composition according to claim 7, further comprising polysaccharides, inorganic components, hydrolyzed peptides, carbohydrates, lipids, and micro/macro elements.

19. The agricultural composition according to claim 16, wherein said keratin, collagen or chitosan are derived from bio-waste.

20. The method according to claim 8, wherein said nanoparticle has a pore network which releases the hydrolyzed peptides by diffusion.

21. A method for reducing the amount of fertilizer applied to a crop, comprising applying a nanoparticle composition to said crops, wherein said nanoparticles have an anchoring peptide on their surface which promotes adhesion to the plant and wherein said nanoparticles comprise hydrolyzed peptides.

22. The method according to claim 21, wherein said nanoparticle composition further comprises nanocomposites, pesticides, herbicides, bactericides, fungicides, insecticides, acaricides, miticides, nemanticides, molluscicides, growth regulators, sources of nitrogen, phosphorus and/or potassium, carbohydrates, magnesium, calcium, sulfur, and/or their precursors in the form of metal salt or a nanoparticle, and metals.

23. The method according to claim 21, wherein said nanoparticles include a molecule which releases the hydrolyzed peptides in response to changes in the environment, and/or enzymatically.

24. The method according to claim 23, wherein said molecule which releases the hydrolyzed peptides is a pH responsive molecule, a temperature responsive molecule, an enzymatically responsive molecule, and/or a sonication responsive molecule.

25. A method for recycling bio-waste into a composition suitable for use in consumer products, comprising:
a) selecting a bio-waste based on the intended application,
b) mixing the bio-waste with at least one natural eutectic solvent to extract at least one biopolymer selected from the group consisting of peptides, polysaccharides and lipids,
c) reducing the size of the extracted bioactive peptides to produce nanopeptides of different sizes and shapes,
d) adding at least one additional bioactive agent during step c) to produce a chain bio-polymer delivery system and/or an encapsulated nano bio-polymer delivery system, and
e) formulating the chain bio-polymer delivery system and/or encapsulated nano bio-polymer delivery system with suitable carriers, wherein said consumer product is selected from the group consisting of a cosmetic, cosmeceutical, perfume, food, and biofuel.
